SEQUENCE LISTING (44 sequences)

SEQ ID NO: 1 - protein, 122 aa, Homo sapiens
QVQLVQSGAEVKKSGASVKVSCQASGYTFTDYWMHWVRQAPGQGLEWMGIIDGSGGSTNSAQKFRGRLTMTRDTSTRTVYMELSSLRSDDTAVYYCARVMTPKFTYDAFEIWGQGTVVTVSS

SEQ ID NO: 2 - protein, 127 aa, Homo sapiens
QVQLVESGSEVKKPGASVKVSCKASGYDFSRHGITWVRQAPGQGLEWMETGWISGYDGNTNYAQRLRGRVTMETTTDTSTSTVYMETELRSLRSDDTAVYYCARDRFVEWFIVIDFWGQGTLVTVSS

SEQ ID NO: 3 - protein, 141 aa, Homo sapiens
EVQLVESGGGLVQPGGSRKLSCAASGFTFSSYGMHWVRQAPEKGLEWVAYISSSSGTIYYADTVKGRFTISRDNAKNTLFLQMTSLRSEDTAMYYCARHTMSNSYCELKLKPPAAAPGNVHRLQKGEFQHWGQGTLVTVSS

SEQ ID NO: 4 - protein, 130 aa, Homo sapiens
QVQLVESGGGVVQPGGSLRLSCAASGFTFSSYGMETHWVRQAPGKGLEWVAFIWYDGNNKYYVDSVRGRFTISRDNFKNMETLYLQMETTGLRGEDTAVYYCAKDLRATAAAWFGVPSVWGQGVLVTVSS

SEQ ID NO: 5 - protein, 122 aa, Homo sapiens
QVQLLESGPGQVKPSETLSLTCTVSGASITCDSCNWDWIRQSPGKGLEWIGNHIFYSGRTYHKYNPSLESRVTIAVDTSKNQFSLRLTAVTAADTAVYYCARTVHGVATDRWGQGTLVTVSS

SEQ ID NO: 6 - protein, 141 aa, Homo sapiens
EVQLVESGGDLVKPGGSLKLSCAASGFTFSSYGMSWVRQTPDKRLEWVATISSGGSYTYYPDSVKGRFTISRDNAKNTLYLQMSSLKSEDTAMYYCARHTMRKCYCELKLKPPAAAPGNVHRLQKGEFQHWGQGTLVTVSS

SEQ ID NO: 7 - protein, 139 aa, Homo sapiens
EVQLVESGGGLIQPGRSLKLSCTASGFTFGDYVMETGWVRQAPGKGLEWVGFIRSKAYGGTIAGTTEYAASVRGRFTISRDDSKSIAYLQMETNSLKTEDTAVYYCTRGSCSITSCAPEVLYGMETDVWGQGTTVTVSS

SEQ ID NO: 8 - protein, 121 aa, Homo sapiens
EVQLVESGGGSVQPGRSLRLSCAASGFTFDDYAMQWVRQAPGKGLEWVAGLSWNGGTIGYADSVKGRFTVSRDNAKNSLYLQMNSLRAEDTALYYCVKDMRYNWNAGLDYWGQGTLVTVSS

SEQ ID NO: 9 - protein, 125 aa, Homo sapiens
QVQLVESGGGVVQPGGSLRLSCAASGFPFSSFGLHWVRQAPGKGLEWVSFISYDGTNQYYGDFVRGRFTISRDNSKNTVFLQMNSLRAEDTAVYYCAKETITMVPGSFAHYVDFWGKGTTVTVSS

SEQ ID NO: 10 - protein, 122 aa, Homo sapiens
QVQLQESGPGLVKPSETLSLTCTASGGSISSSDYYWGWIRQSPGKGLEWIANIYYGGSTYYNPSLRSRVSISIDTSKNQFSLQMGSLTAADTAIYYCARLNDITVVGPWDKWGPGTLVTVSS

SEQ ID NO: 11 - protein, 120 aa, Homo sapiens
QVQLQESGPGLVKPSETLSLTCTVSGSSISNYYWSWIRQSPGKGLEWIGFIYYGGNTKYNPSLKSRVTISQDTSKSQVSLTMSSVTAAESAVYFCARASCSGGYCILDYWGQGTLVTVSS

SEQ ID NO: 12 - protein, 129 aa, Homo sapiens
QVQLVQSGGGVVQPGRSLRLSCAASGFTFSTYGMHWVRQAPGKGLEWVAVISYDGNYKYYADSVKGRFTISRDNSKNTLYLEMNSLRTEDTALYYCAKDSQLRSLLYFDWLSQGYFDHWGQGTLVTVSS

SEQ ID NO: 13 - protein, 122 aa, Homo sapiens
QVQLVESGPGLVKPSETLSLTCTASGGSISSSDYYWGWIRQSPGKGLEWIANIYYGGSTYYNPSLRSRVSISIDTSKNQFSLQMGSLTAADTAIYYCARLNDITVVGPWDKWGPGIQVTVSS

SEQ ID NO: 14 - protein, 114 aa, Homo sapiens
QVQLVQSGGGVVQPGRYLRLSCAASTFTFSSNNMWVRQAPGKGLEWVALISYDGRISRDKSKKTVYLQMSSLRDEDTAVYKAGSTVKKRDMMKTREMINCFDPWGRGTLVTVSS

SEQ ID NO: 15 - protein, 119 aa, Homo sapiens
EVQLVESGGGLVQPGRSLRLSCVASGFTFGDYAMHWVRQGPGKGLEWVSGINGNSDSVGYADSVKGRFTVSRDNAKNSLYLQLNSLTVEDTALYYCAKDLSWGEAFDIWGQGTMVTVSS

SEQ ID NO: 16 - protein, 128 aa, Homo sapiens
EVQLVESGGGLVQPGRSLRLSCATSGFTFYDYAMYWVRQAPGKGLEWVGFIRSQRYGGTSEYAASVKGRFTISRDDSKTIVYLQMNSLQAEDTAVYYCTRGSYRCTLTACYPGYLDYWGQGTLVTVSS

SEQ ID NO: 17 - protein, 130 aa, Homo sapiens
QVQLVQESGPGLVKPSQTLSLTCTVSGGSVNSGAYSWNWIRQPAGKGLEWIGRIDGRGSTKYNPSLKSRVTMSIDTSNKQFSLKLTSVTAADTAVYYCATTAVRSKFGVIVQNAYWFDPWGQGTLVTVSS

SEQ ID NO: 18 - protein, 126 aa, Homo sapiens
QVQLLESGGGVVQPGRSLRLSCTASGFTFSSYAMHWVRQAPGKGLEWVAVMSSDGKNKYYADSVKGRFTVSRDNSKNTLYLQMDSLRPEDTAVYYCAREGKIESGELDYYFGMDVWGQGTTVTVSS

SEQ ID NO: 19 - protein, 130 aa, Homo sapiens
QVQLVESGPGLVKPSQTLSLTCTVSGDSISGGRYYWSWLRQPAGKGLEWIGRIHASGRTKYKPSLESRVTISVDTSNNQFSLKLTSLTAADTAVYYCARGPTPYTYDSGGLYYEEYFQSWGQGTLVTVSS

SEQ ID NO: 20 - protein, 113 aa, Homo sapiens
DIQMTQSPFSLAVSLGDRATINCKSSQTVFFSYNNKNSVAWYQQKPGQPPKLLIYWASTRVSGVPERFSGSDSGTDFTLTISSLHAEDVAVYYCQQYFTNSPTFGQGTKVEIK

SEQ ID NO: 21 - protein, 112 aa, Homo sapiens
DIVMTQSPLSLAVTPGEPASISCRSSQSLLQSNGYNYLDWYLQKPGQSPQLLIYWGSNRASGVPDRFSGSGSGTDFTLKITRVEAEDVGVYYCMQALQTPLTFGQGTKVEIK

SEQ ID NO: 22 - protein, 106 aa, Homo sapiens
DIQMTQSPFTLSASVGDRVTITCRASQPIDKWLAWFQQKPGKAPNLLIYKASTLDSGVPSRFSGSGSGTEFTLTISSLQPDDFATYYCQQYNRYWTFGPGTKVEIK

SEQ ID NO: 23 - protein, 112 aa, Homo sapiens
DIVMTQSPLSLSVTPGQPASIFCKSNQSLLHNDDKTYLYWYLQKPGQSPHLLIYELSSRFSGVPDRFSGSGSGTDFTLRISRVEAEDVGIYYCMQGIMLPPTFGQGTKVEIK

SEQ ID NO: 24 - protein, 107 aa, Homo sapiens
EIVLTQSPFTLSLSPGETATLSCRASKSVSIYLAWYQQKPGQAPRLLIYDASNRATGIPARFSGSGSGTVFTLTITSLEPEDSAVYFCQHRDNWRGTFGPGTKVEIK

SEQ ID NO: 25 - protein, 106 aa, Homo sapiens
DIQMTQSPFSLSASVGDRVTITCRASQPIDKWLAWFQQKPGKAPNLLIYKASTPDSGVPSRFSGSGSGTEFTLTIGSLQPDDFATYYCQQYNRYWTFGPGTKVEIK

SEQ ID NO: 26 - protein, 107 aa, Homo sapiens
AIQLTQSPSSLSASVGDRVTITCRASQGISSALTWYQQKPGKTPKLLIYDASRLESGVPSRFSGSASGTDFTLTISSLQPEDFATYYCQHFNTFPLTFGGGTKVEIK

SEQ ID NO: 27 - protein, 107 aa, Homo sapiens
EIVLTQSPSTLSLSPGETATLSCRASKSVSIYLAWCQQKPGQAPRLLIYDASNRATGIPARFSGSGSGTVFTLTITSLEPEDSAVYFCQHRDNWRGTFGPGTKVEIK

SEQ ID NO: 28 - protein, 109 aa, Homo sapiens
QSALTQPPSVSGAPGQRVTISCTGSSSNIGAGYDVHWYQQLPGTAPKLLIYGNNNRPSGVPDRFSGSKSGTSASLAITGLQAEDEADYYCQSYDSTCYVFGTGTKVTVL

SEQ ID NO: 29 - protein, 110 aa, Homo sapiens
QSVLTQPPSASGTPGQRVTISCSGRYSNIGSNYVYWYQQLPGTAPKLLIYRNNERPSGVPDRFSGSRSGTSASLAISGLRSEDEADYYCAAWDDSLSGGVFGGGTKLTVL

SEQ ID NO: 30 - protein, 109 aa, Homo sapiens
QSVLTQPPSVSGAPGQRVTISCTGSSSNIGAGYDVHWYQQLPGTAPKLLIYGNNNRPSGVPDRFSGSKSGTSASLAITGLQAEDEADYYCQSYDSTCYVFGTGTKVTVL

SEQ ID NO: 31 - protein, 111 aa, Homo sapiens
QSALTQPRSVSGSPGQSVTISCTGTSSDVGGYNYVSWYQQHPGKAPKLMIYAVSKRPSGVPDRFSGSKSGNTASLTISGLQAEDEADYYCCSYVASYTFWVFGTGTKVTVL

SEQ ID NO: 32 - protein, 110 aa, Homo sapiens
QSVLTQPPSVSGAPGQRISISCTGTSSNIGEGYDVHWYQKIPGRAPKLLIYGNFNRPSGVPDRFSGSKSGTSASLTITGLQAEDEADYYCQSYDISLTVIFGGGTKVTVL

SEQ ID NO: 33 - protein, 112 aa, Homo sapiens
QSALTQPRSVSGSPGQSVTISCTGTSSNVGDYKYVSWYQQHPGKAPKLIIYNVSKRPAGVPNRFSGSKSGNTASLTISGLQADDEADYYCSSYAGSNSLNVIFGGGTKLTVL

SEQ ID NO: 34 - protein, 113 aa, Homo sapiens
QSALTQPRSVSGSPGQSVTISCTGNSSDIGGYNFVSWYQQHPGKVPKLIIFDVNKRPSGVPDRFSGSRSANTASLTISGLQAEDEADYYCCSYAGSDTSERAVFGGGTKVTVL

SEQ ID NO: 35 - protein, 106 aa, Homo sapiens
SYELTQPLSVSVALGQTARITCGGNNIGSKNVHWYQQKPGQAPVLVIYRDNNRPSGIPERFSGSNSGNTATLTISRAQAGDEAEYYCQVWDSRIYVFGSGTKVTVL

SEQ ID NO: 36 - protein, 106 aa, Homo sapiens
SYELTQPPSVSVSPGQTANITCSGDKLVDKYVCWYQVRPGQSPVLVIYSDKKRPSGIPERISGSNSGNTATLTISGSQAMDEADYYCQAWDSSIVVFGGGTKLTVL

SEQ ID NO: 37 - protein, 96 aa, Homo sapiens
VQLVESGGGVVQPGGSLRLSCAASGFTFSSYGMHWVRQAPGKGLEWVAFIRYDGSNKYYADSVKGRFTISRDNSKNILYLQMNSRAEDTAVYYCAK

SEQ ID NO: 38 - protein, 97 aa, Homo sapiens
VQLVESGGGVVQPGRSLRLSCAASGFTFSSYAMHWVRQAPGKGLEWVAVISYDGSNKYYADSVKGRFTISRDNSKNTLYLQMNSLRAEDTAVYYCAR

SEQ ID NO: 39 - protein, 124 aa, Artificial sequence
  Consensus VH chain
  misc_feature (98)..(99): Xaa can be any naturally occurring amino acid
  misc_feature (101)..(104): Xaa can be any naturally occurring amino acid
  misc_feature (108)..(110): Xaa can be any naturally occurring amino acid
  misc_feature (113)..(113): Xaa can be any naturally occurring amino acid
  misc_feature (116)..(116): Xaa can be any naturally occurring amino acid
VQLLESGGGVVQPGGSLRLSCAASGFTFSSYGMHWVRQAPGKGLEWVAFISYDGSNKYYADSVKGRFTISRNSKNTLYLQMNSLRAEDTAVYYCAKEXXIXXXXGSFXXXVDXWGXGTTVTVSS

SEQ ID NO: 40 - protein, 735 aa, adeno-associated virus 2 [sequence omitted]

SEQ ID NO: 41 - protein, 736 aa, adeno-associated virus 3
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 42 - protein, 736 aa, adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 43 - protein, 738 aa, adeno-associated virus 8
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 44 - protein, 738 aa, adeno-associated virus rh10
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

(19) United States
(12) Patent Application Publication
(10) Pub. No.: US 2018/0179265 A1
(43) Pub. Date: Jun. 28, 2018

(54) ENGINEERED HUMAN ANTI-AAV ANTIBODIES AND USES THEREOF

(71) Applicant: The Trustees of the University of Pennsylvania, Philadelphia, PA (US)

(72) Inventors: April Giles, Philadelphia, PA (US); James M. Wilson, Philadelphia, PA (US)

(21) Appl. No.: 15/564,680

(22) PCT Filed: Apr. 26, 2016
(86) PCT No.: PCT/US2016/029374

§ 371 Date: Oct. 5, 2017

Related U.S. Application Data
(60) Provisional application No. 62/153,000, filed on Apr. 27, 2015.
(60) Provisional application No. 62/323,740, filed on Apr. 17, 2016.

(51) Int. Cl. C07K 16/08; C12N 7/00; G01N 33/68; C12Q 1/68

(52) U.S. Cl. CPC C07K 16/081; C12N 7/00; G01N 33/6854; C12Q 1/6876; C07K 2317/14; C07K 2317/565; C07B 2200/11; C12N 2750/14151; C12N 2750/14143; C07K 2317/21

(57) ABSTRACT

A method for generating engineered human anti-AAV antibodies is described. Also described are compositions, panels and arrays containing these antibodies and uses thereof.

### STATEMENT REGARDING FEDERALLY SPONSORED RESEARCH OR DEVELOPMENT

This work was supported in part by grants from the National Institutes of Health, No. 5P01-HD-05247. The US government may have certain rights in this invention.

### INCORPORATION-BY-REFERENCE OF MATERIAL SUBMITTED IN ELECTRONIC FORM

Applicant hereby incorporates by reference the Sequence Listing material filed in electronic form herewith. This file is labeled “UPN_15_7424PCT_ST25.txt”.

### BACKGROUND OF THE INVENTION

Over the past decade, adeno-associated virus (AAV)-based vectors have gained attention in both basic, preclinical and clinical research and AAV vectors are now among the most promising vector systems for gene therapy applications. K. Rapti, et al, Molecular Therapy (2012); 20 1, 73-83. This is partially due to the fact that AAV is not associated with any known human disease, that AAV vectors can, at least in nondividing cells, generate long-term transgene expression—even in the absence of genome integration—and that AAVs display relatively low immunogenicity. The relatively low immunogenicity notwithstanding, it has been recognized that the high prevalence of neutralizing antibodies against some AAVs in the human population presents a considerable obstacle to the broad use of AAV vectors in clinical gene therapy. S. Boutin, et al, (2010). Prevalence of serum IgG and neutralizing factors against adeno-associated virus (AAV) types 1, 2, 5, 6, 8, and 9 in the healthy population: implications for gene therapy using AAV vectors. Hum Gene Ther 21: 704-712; Calcedo, R, et al., (2009). Worldwide epidemiology of neutralizing antibodies to adeno-associated viruses. J Infect Dis 199: 381-390; van der Marel, S, et al, Petry, H et al. (2011). Neutralizing antibodies against adeno-associated viruses in inflammatory bowel disease patients: Implications for gene therapy. Inflamm Bowel Dis (epub ahead of print).

Neutralizing antibodies against AAV, whether generated via natural exposure or exposure to the viral vector, have been shown to significantly reduce the efficiency of gene transfer, particularly following intravenous administration. One proposed approach to overcoming this obstacle is the engineering of an AAV capsid that is able to evade neutralization by these antibodies. See, e.g., discussion in Jeune V L, Joergensen J A, Hajjar R J, Weber T. “Pre-existing Anti-Adeno-Associated Virus Antibodies as a Challenge in AAV Gene Therapy”. Human Gene Therapy Methods. 2013; 24(2):59-67] The epitopes of a handful of antibodies have been mapped to their respective AAV capsids via cryo-EM or X-ray crystallography [see, e.g., Gurda et al, “Capsid Antibodies to Different Adeno-Associated Virus Serotypes Bind Common Regions”. J Virol, August 2013, 87(16):9111-24, epub 2013 Jun. 12; Y S Tseng et al, J Virol, February 2015; 89(3): 1794-808, Epub 2014 Nov. 19; B L Gurda et al, J Virol, August 2012; 66(15): 7739-51; Epub 2012 May 16], but these antibodies are most often mouse monoclonals and their relevance to human vector immunology has not been identified. In addition, these antibodies likely represent only a small fraction of the humoral response to AAV, as no comprehensive panel against a single serotype has been studied; rather, only one or two quality capsid:Fab structures have been obtained for a given serotype. See, e.g., Gurda B L, et al. 2013, cited above.

Recently, Wardemann and Kofer have described expression cloning of human B cell immunoglobulins [Chapter 5, R. Kuppers (ed.) Lymphoma: Methods and Protocols, Methods in Molecular Biology, vol. 971, pp. 93-111 (2013)]. J. Huang et al, have described feeder cells useful in isolation of human monoclonal antibodies from peripheral blood B cells [Nature Protocols, Vol. 8 No. 10 (2013), p 1907-1915].

What are needed are more efficient techniques for generating human antibodies to a selected AAV capsid and novel human anti-AAV antibodies.

### SUMMARY OF THE INVENTION

The present invention provides novel human anti-AAV immunoglobulins which are useful for a variety of clinical, purification, and research uses, methods of obtaining same, and uses thereof.

In one aspect, a method is provided for identifying human anti-AAV immunoglobulin peptides and polypeptides, and for generating engineered human anti-AAV antibodies containing these peptides and polypeptides. This method involves: a) sorting memory B cells; (b) culturing the memory B cells and screening supernatant from the cell culture for anti-AAV activity and/or binding to AAV; (c) amplifying immunoglobulin variable domains selected from one or more of a heavy chain and/or a light chain, or a fragment thereof; (d) sequencing amplified immunoglobulin variable domains, deducing amino acid sequences, and designing nucleic acid sequences encoding the deduced amino acids of the anti-AAV variable region(s); (e) cloning de novo synthesized immunoglobulin variable domains which were backtranslated from the amino acid sequences and optimized (e.g., using optimal codon adaptation tables and/or other techniques) to generate sequences coding for full-length human monoclonal antibodies; (f) deconvoluting (matching) heavy/light chain pairs, and g) identifying the binding epitopes for each antibody. In one embodiment, a human anti-AAV antibody is provided which binds to a common epitope present on multiple AAVs. In another embodiment, a human anti-AAV antibody is provided which binds selectively to a specific AAV capsid.

In another aspect, a panel of human anti-AAV antibodies is provided, wherein said anti-AAV antibodies are generated according to the methods provided herein.

In still another embodiment, an engineered antibody is provided which comprises an anti-AAV heavy chain comprising a heavy chain sequence provided herein, or a fragment thereof and a heterologous sequence. In still a further aspect, an engineered antibody comprises an anti-AAV kappa light chain comprising a kappa light chain sequence provided herein, or a fragment thereof and a heterologous sequence. In yet a further embodiment, an engineered antibody comprising an anti-AAV lambda light chain is provided which comprises a lambda light chain sequence described herein, or a fragment thereof and a heterologous sequence.

In another embodiment, the invention provides an engineered immunoglobulin or other moiety comprising a human anti-AAV heavy chain and/or light chain complementarity determining region (CDRs). In one embodiment, the engineered immunoglobulins contain framework regions from a source which differs from the source of CDR.

In another embodiment, the invention provides synthetic and recombinant nucleic acid sequences encoding the anti-AAV CDRs and immunoglobulins.

In still a further embodiment, a solid support is provided which comprises one or more of the anti-AAV immunoglobulins generated as described herein.

In yet another embodiment, a method of purifying an AAV vector is provided. The purification may comprise contacting a suspension comprising a selected AAV with the solid support which comprises one of more immunoglobulins generated as described herein.

In addition, the antibodies provided herein are useful for a variety of purposes including, without limitation, for the evaluation of neutralizing or binding capacity, for epitope mapping to identify common and unique immunodominant regions of the AAV capsids for monitoring the presence of AAV capsid following delivery of a viral vector and for selecting AAV capsid for repeat vector administrations.

In addition, in addition to full-length or intact anti-AAV antibodies, various CDRs of the immunoglobulin sequences provided herein may be used to engineer other antibodies or other moieties. Thus, provided herein are immunoglobulins and other moieties which contain functional anti-AAV immunoglobulin sequences.

Still other aspects and advantages of the invention will be readily apparent from the following detailed description of the invention.

### BRIEF DESCRIPTION OF THE DRAWINGS

FIG. 1 is a flow chart illustrating one embodiment of the B cell cloning workflow described herein. The optional characterization step illustrated in the figure represents (a) assessing for binding to the 5-fold symmetry on an AAV capsid, (b) assessing for binding to the 3-fold symmetry on an AAV capsid and/or (c) a docking model of the surface complex between the antibody and its epitope on the AAV capsid.

FIG. 2 provides a sample alignment of clones utilizing the same or similar germline loci [VH3-30*3, SEQ ID NO: 37; VH 3-30*7, SEQ ID NO: 38]. In this example, 2.46 D10H [SEQ ID NO: 4], 2.47 D11H [SEQ ID NO: 3], and 2.81 C10H all most closely align to the VH 3-30*2 germline gene. 272D3H [SEQ ID NO: 9] and 2.81 C10H [SEQ ID NO: 7] sequences are nearly identical, while 2.46 D10H [SEQ ID NO:4] varies at a number of positions and is more similar to the germline sequence, suggesting a lesser degree of affinity maturation. The resulting phylogenetic tree is also shown, demonstrating the relatedness of these clones.

FIGS. 3A-3B provide a sequence alignment (VP1 numbering) of AAV2 [SEQ ID NO:40], AAV3B [SEQ ID NO: 41], AAV9 [SEQ ID NO: 42], AAV8 [SEQ ID NO: 43], and AAVrh10 [SEQ ID NO: 44] (in order of sequence homology); a consensus sequence is shown in SEQ ID NO: 39. Shading is indicative of homology of amino acid identity at a given position, assigned by Vector NTI default settings. Boxed residues are residues predicted to interact with the indicated antibody.

FIGS. 4A-4D illustrate serotype binding preferences for anti-AAV antibodies. A. Antibodies were expressed in 293 cells and screened for binding to AAV2, AAV3B, AAV9, AAV8, and AAVrh10 by ELISA. Binding reported as absorbance when bound to 3B:absorbance when bound to secondary serotype, AAV3B binding reported as 1 for each antibody. Antibodies with predicted binding residues are displayed as black-filled icons. Serotypes are displayed on X-axis in order of amino acid identity as determined by AAV variant phylogeny. FIG. 4B to FIG. 4D Labeled binding preference for each anti-AAV antibody, broken into separate graphs for visualization purposes.

### DETAILED DESCRIPTION OF THE INVENTION

The invention provides methods for generating fully human anti-AAV immunoglobulins, including engineered anti-AAV immunoglobulins. These immunoglobulins may be used individually or in collections or panels. These immunoglobulins and fragments thereof, may also be produced synthetically in whole or in part or expressed recombinantly. A variety of uses for the immunoglobulin constructs for AAV vector development, production, purification, monitoring, identity analyses, and for diagnostic purposes will be readily apparent to one of skill in the art. The immunoglobulins may also be used to generate anti-idiotypic antibodies which are useful for these purposes, and optionally also in therapeutic and immunomodulatory regimens.

The term “immunoglobulin” is used herein to include intact or whole antibodies and functional fragments thereof, as defined below. Immunoglobulins may exist in a variety of forms including, for example, monoclonal antibodies, camelized single domain antibodies (the heavy chain of the antibodies have lost a constant region (CH1) which is replaced by an extended hinge region), polyclonal antibodies, intracellular antibodies (“intrabodies”), recombinant antibodies, multispecific antibody, antibody fragments, such as, Fv, Fab, F(ab)₂, F(ab)₃, Fab′, Fab′-SH, F(ab′)₂, single chain variable fragment antibodies (scFv), tandem/bis-scFv, Fc, pFc′, scFvFc (or scFv-Fc), disulfide Fv (dsfv), bispecific antibodies (bc-scFv) such as BiTE antibodies; camelid antibodies, resurfaced antibodies, humanized antibodies, fully human antibodies, single-domain antibody (sdAb, also known as NANOBODY®), chimeric antibodies, chimeric antibodies comprising at least one human constant region, and the like. “Antibody fragment” refers to at least a portion of the variable region of the immunoglobulin that binds to its target, e.g., the AAV capsid. As used herein the term “full-length”, “intact”, or “whole” antibody refers to antibodies which have both two immunoglobulin (Ig) heavy chains and two Ig light chains. In humans, there are two types of light chains, i.e., the kappa (κ) chain, encoded by the immunoglobulin kappa locus (IGK) on chromosome 2 and the lambda (λ) chain, encoded by the immunoglobulin lambda locus (IGL) on chromosome 22. Antibodies are produced by B lymphocytes, each expressing only one class of light chain. Once set, light chain class remains fixed for the life of the B lymphocyte. In general, in a healthy individual, the total kappa to lambda ratio is roughly 3:1 in serum (measuring intact whole antibodies) or 1:1.5 if measuring free light chains. However, other ratios may be determined by one of skill in the art.

A “functional fragment” of an antibody as described herein refers to a protein which is not an intact antibody, but which is capable of binding to a desired target, e.g., an epitope on an AAV capsid, with sufficient binding affinity to affect a desired result. In one embodiment, a “functional fragment” may be one or more of the complementarity determining regions (CDRs) of an anti-AAV immunoglobulin chain or one or more CDRs engineered into a constant region framework which is from a different source. An immunoglobulin contains a “framework region” which is a region in the variable domain of an immunoglobulin which is exclusive of the complementarity determining regions (CDRs). In general, each antibody chain contains 4 framework regions separated by 3 CDRs. “CDRs” are part of the variable chains in immunoglobulins where these molecules bind to their ligand (e.g., the AAV capsid). There are three CDRs (CDR1, CDR2, and CDR3) on a full-length immunoglobulin chain.

As used herein, an “immunoglobulin domain” refers to a domain of an antibody heavy chain or light chain as defined with reference to a conventional, full-length antibody. More particularly, a full-length antibody contains a heavy (H) chain polypeptide which contains four domains: one N-terminal variable (VH) region and three C-terminal constant (CH1, CH2 and CH3) regions and a light (L) chain polypeptide which contains two domains: one N-terminal variable (VL) region and one C-terminal constant (CL) region. An Fc region may contain two domains (CH2-CH3) or three domains (CH1-CH2-CH3). A Fab region may contain one constant and one variable domain from each the heavy and light chain.

The term “neutralizing antibody”, abbreviated “NAb” is an immunoglobulin (including a functional fragment as defined herein) which defends a cell from an antigen or infectious body by inhibiting or neutralizing any detectible effect it has biologically. A variety of assays are known for determining whether an antibody is a neutralizing antibody. See, e.g., R Calcedo et al, J Infect Dis, 2009 Feb. 1; 199(3): 381-390.

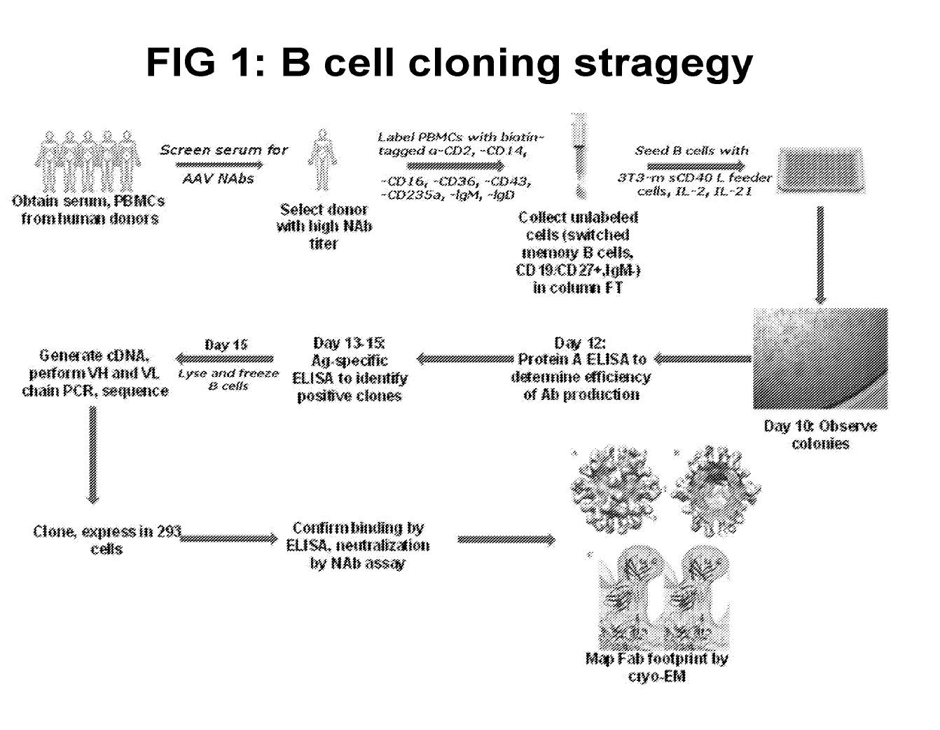

One embodiment of the method for generating anti-AAV antibodies is illustrated in FIG. 1. From the following discussion, variations on the technique in FIG. 1 which are encompassed within the invention will be readily understood by one of skill in the art.

To obtain human immunoglobulins against AAV (which may be abbreviated hu anti-AAV Ig), e.g., hu anti-AAV monoclonal antibodies, serum samples from human donors are screened for neutralizing antibodies against the target AAV and donors are selected with the highest antibody titer. See, e.g., R Calcedo et al, J Infect Dis, 2009 Feb. 1; 199(3): 381-390. Typically donors with the highest neutralizing antibody titer will be selected. Peripheral blood mononuclear cells (PBMCs) from these patients will be purified and labelled, e.g., with biotin-tagged α-CD2, -CD14, -CD16, -CD36, -CD43, -CD235a, IgM, -IgD to remove unwanted cells.

As used herein, “switched memory B cells” refer to memory B cells which are IgM negative, i.e., in order to exclude naïve B cells. Switched memory B cells are obtained and seeded onto feeder cells. Optionally, the switched memory B cells are bulk sorted using magnetic beads. As used herein, the term “magnetic-activated cell sorting (MACS)” is a method for separation of various cell populations depending on their surface antigens (CD molecules) invented by Miltenyi Biotec. The name MACS is a registered trademark of the company. Magnetic beads may be obtained from commercial sources (Miltenyi Biotech). However, other methods for sorting switched memory B cells may be substituted for the magnetic beads including, e.g., other solid phase moieties, flow cytometry, or antigen-specific sorting (i.e., labelled AAV for selecting AAV-specific cells). These sorting methods may utilize either negative selection or positive selection and the appropriate fraction is selected for seeding onto feeder cells. For example, for switched memory B cells may be identified by the characteristics of being CD19/CD27+ and IgM- in a flow-through column.

One example of suitable feeder cells includes the irradiated 3T3-msCD40L cells (J. Huang, 2013 et al, cited above) grown in the presence of suitable growth factors (e.g., in the presence of IL-2 and IL-21) as described in the working examples. Alternatively, a mix of growth factors and antibodies may be used in the place of the feeder cells [M Wiesner al, (2008) PLoS ONE, 3(1):e1464; E L Carpenter, et al, J Translational Medicine, 2009, 7(9): 93]. The memory B cells are cultured for a sufficient length of time to promote expansion and antibody secretion, e.g., for about 8-12 days. Culture supernatants are screened for reactivity with the target AAV, e.g., approximately at days 10-12. In one embodiment, reactivity is assessed with a suitable enzyme-linked immunoassay (ELISA). Suitable ELISA formats may include those designed to target human IgG1, e.g., Protein A ELISA, Protein G ELISA, Protein L ELISA, and are commercially available [e.g., as kits from Life Technologies; Repligen Corp; Abcam; Enzo Life Sciences, among others] and have been described in the literature. At approximately days 10-15, clones may be selected for specific reactivity, i.e., reactivity with a single AAV capsid, reactivity with a small defined set of AAV (e.g., 2-3 different AAV, such as only AAV2 and AAV3B), or reactivity with a larger group of AAV (e.g., 4-12 different AAV, or more). In general, a clone having specificity for one or more AAV capsids will be selected.

Once supernatants with the desired activity are selected, the cells are harvested and RNA is extracted and subjected to RT-PCR to amplify the anti-AAV immunoglobulins. The sequences targeted for being amplified are at a minimum, the variable domain of the heavy chain immunoglobulin (VH). In addition, it will generally be desired to amplify the variable light chain (VL) of the immunoglobulins, optionally with separate sets of primers designed for both a kappa and/or a lambda chain. An example of a suitable technique is provided in Wardemann and Kofer Chapter 5, R. Kuppers (ed.) Lymphoma: Methods and Protocols, Methods in Molecular Biology, vol. 971, pp. 93-111 (2103), which is incorporated herein by reference. Due to variances in the number of cells that are used for cDNA synthesis in the present method, the amount of cDNA used as a template in the 1^{st} PCR may need to be adjusted from that described in Wardemann and Kofer, as described in the working examples below. In order to obtain variable heavy and variable light chain sequences for expression and characterization of these antibodies, a nested PCR can be performed based on conserved regions in the immunoglobulin genes. In the working example provided below, the published primers for the immunoglobulin variable heavy chain (VH) and variable portions of the Vκ, or Vλ light chains were used. However, one of skill in the art may design and/or select other primers for use in the method of the invention which allow for separate application of heavy chain and/or light chain immunoglobulins, or at least the variable regions thereof, or at least the constant regions thereof. Optionally, the method may be performed to isolate only the heavy chain. Subsequent to the first and second PCR rounds, the coding sequences for at least the immunoglobulin(s) are obtained. While these coding sequences may be used for the following cloning steps, in one embodiment, the PCR-amplified sequences are used to generate artificial sequences. More particularly, in one embodiment, the amino acid sequences encoded by the amplified immunoglobulins (e.g., the heavy chain variable, light chain kappa variable, and/or the light chain lambda sequence) may be deduced from the amplified coding sequence using conventional codon translation charts. From the deduced immunoglobulin polypeptide sequences, nucleic acid sequences may be synthesized de novo and used to express the anti-AAV immunoglobulins in a suitable cell line. Thus, the nucleic acid sequences used to express the human anti-AAV antibodies may differ significantly from the wild-type human immunoglobulin sequences obtained from the PCR (e.g., up to about 30% divergent, or about 5% to about 25% divergent). Suitably, this method permits production of larger scale amounts of anti-AAV antibodies. In one embodiment, the synthesized nucleic acid sequences are codon optimized. Codon-optimized coding regions can be designed by various different methods. This optimization may be performed using methods which are available on-line, published methods, or a company which provides codon optimizing services. One codon optimizing method is described, e.g., in WO 2015/012924, published Jan. 29, 2015, and the documents cited therein, which are incorporated by reference herein. Briefly, the nucleic acid sequence encoding the product is modified with synonymous codon sequences. Suitably, the entire length of the open reading frame (ORF) for the product is modified. However, in some embodiments, only a fragment of the ORF may be altered. By using one of these methods, one can apply the frequencies to any given polypeptide sequence, and produce a nucleic acid fragment of a codon-optimized coding region which encodes the polypeptide.

Optionally, the immunoglobulins may be further characterized. Such characterization may take the form of mapping the Fab footprint by cryo-EM [Gurda et al, J Virol, August 2013, 87(16):9111-24, epub 2013 Jun. 12; Y S Tseng et al, J Virol, February 2015; 89(3): 1794-808, Epub 2014 Nov. 19; B L Gurda et al, J Virol, August 2012; 66(15): 7739-51; Epub 2012 May 16]; docking modelling, and/or homology modelling. In one embodiment, immunoglobulins which bind to the 5-fold axis of symmetry on the AAV capsid are preferentially selected. In another embodiment, immunoglobulins which bind to the 3-fold axis of symmetry on the AAV capsid are preferentially selected.

In one embodiment, the nucleic acid sequences of the anti-AAV immunoglobulins (whether native or synthetic) are engineered into shuttle (plasmid) constructs containing heterologous heavy chain and/or light chain constant regions. Alternatively, these anti-AAV immunoglobulin binding domains may be co-expressed with constructs separately providing the constant regions. Suitably, such constant regions are from human antibodies from another source. While such antibodies may be from an anti-AAV antibody, more typically, the constant regions are provided from a non-AAV antibody. Thus, when the heavy chain and/or light chains are expressed, they are chimeric antibodies containing the anti-AAV variable domains. Optionally, these chimeric antibodies may contain, at a minimum, one or more of the CDRs of the anti-AAV antibody obtained according to the techniques provided herein. In one embodiment, the immunoglobulin genes are expressed using TOPO® cloning vectors [available from Life Technologies]. However, a variety of other vectors or techniques may be selected. Optionally, the cloned PCR products (i.e., the heavy chain immunoglobulin, kappa chain, or lambda chain) are sequenced and the respective types of immunoglobulins are aligned in order to generate a consensus sequence for each type of immunoglobulin.

Once obtained, the heavy chain immunoglobulin sequences and/or the light chain sequences may be matched (e.g., by co-expression) to generate an intact antibody, or used to generate engineered intact antibodies by combining a heavy chain or light chain immunoglobulin sequence obtained as described herein with the corresponding light chain or heavy chain from another source, whether it be from another anti-AAV antibody or another type of antibody, or a natural or non-natural source. These intact antibodies, or other immunoglobulins, may be used to generate a panel of human anti-AAV immunoglobulins. In one embodiment, this panel is a collection of human anti-AAV immunoglobulins which are neutralizing. In another embodiment, the panel includes immunoglobulins which are not neutralizing. These panels may contain exclusively immunoglobulins which are specific for a single AAV. Within such a panel, there may be immunoglobulins which recognize different epitopes on that single AAV; alternatively, all of the immunoglobulins may be directed to a single epitope. In another embodiment, these panels may contain immunoglobulins which are specific for a subset of AAVs, e.g., binds only AAV1 and AAV6, but not AAV2, or AAV2 and AAV3B, but not AAV9. In one embodiment, these anti-AAV immunoglobulins recognize multiple AAVs. Suitably, the panels contain at least 3 to 25, or more immunoglobulins, which may be the same or different, e.g., directed against more than one AAV, different types of immunoglobulins directed against the same AAV (recognizing different epitopes, different immunoglobulins recognizing the same epitope, neutralizing, and/or non-neutralizing and/or combinations of these with each other or others). The panel can also be used to evaluate modified AAV capsid and predict if they can evade pre-existing antibody. A variety of other uses will be apparent to one of skill in the art. These panels may be affixed to a solid support, e.g., a multi-well plate, or the like and used for a variety of purposes that will be readily apparent to one of skill in the art. For example, these panels may be used for screening for the presence of AAV and/or assaying AAV levels in a sample (e.g., blood, plasma, or derived from tissue), for diagnosis and/or for monitoring therapy. A single type of a human anti-AAV immunoglobulin may also be bound, or optionally combined with other anti-AAV or other immunoglobulin(s), to a variety of solid supports. Suitable combinations may include, e.g., immunoglobulins directed against more than one AAV, different types of immunoglobulins directed against the same AAV (recognizing different epitopes, different immunoglobulins recognizing the same epitope, neutralizing, and/or non-neutralizing and/or combinations of these with each other or others). Suitable solid supports may include, e.g., a membrane, glass slide, bead, well plate, etc. In one embodiment, a human anti-AAV immunoglobulin may be bound to a bead, or a collection of beads, for purification of a specific AAV, e.g., by affinity purification. In another embodiment, may be well-bound on a suitable plate. In another example, an array may be used for a variety of purposes including, for example, for identified an AAV in a sample.

A variety of different AAV capsids have been described, as have methods for generating AAV vectors have been described extensively in the literature and patent documents, including, e.g., WO 2003/042397; WO 2005/033321, WO 2006/110689; U.S. Pat. No. 7,588,772B2. The source of AAV capsids may be selected from an AAV which targets a desired tissue. For example, suitable AAV may include, e.g., AAV9 [U.S. Pat. No. 7,906,111; US 2011-0236353-A1], rh10 [WO 2003/042397] and/or hu37 [see, e.g., U.S. Pat. No. 7,906,111; US 2011-0236353-A1]. However, other AAV, including, e.g., AAV1, AAV2, AAV3, AAV4, AAV5, AAV6, AAV7, AAV8, [U.S. Pat. No. 7,790,449; U.S. Pat. No. 7,282,199], among others may be selected. However, other sources of AAV may be selected.

In another embodiment, the immunoglobulins generated as described herein may be produced recombinantly, or used to design engineered immunoglobulins. Such engineered immunoglobulins may be contain, e.g., an anti-AAV heavy chain CDR1, CDR2, and/or CDR3, identified by bold and underlining, or the heavy chain variable sequence selected from:

(a) 2.15 G3H (IGHV1-46*01): SEQ ID NO: 1
QVQLVQSGAEVKKSGASVKVSCQASGYTFTDYWMHWVRQAPGQGLEWMGI
IDGSGGSTNSAQKFRGRLTMTRDTSTRTVYMELSSLRSDDTAVYYCARVM
TPKFTYDAFEIWGQGTVVTVSS;
(b) 2.22 C8H (IGHV1-18*01): SEQ ID NO: 2:
QVQLVESGSEVKKPGASVKVSCKASGYDFSRHGITWVRQAPGQGLEWMET
GWISGYDGNTNYAQRLRGRVTMETTTDTSTSTVYMETELRSLRSDDTAVY
YCARDRFVEWFIVIDFWGQGTLVTVSS;
(c) 2.46 C11H (IGHV3-48*01): SEQ ID NO: 3:
EVQLVESGGGLVQPGGSRKLSCAASGFTFSSYGMHWVRQAPEKGLEWVAY
ISSSSGTIYYADTVKGRFTISRDNAKNTLFLQMTSLRSEDTAMYYCARHT
MSNSYCELKLKPPAAAPGNVHRLQKGEFQHWGQGTLVTVSS;
(d) 2.46 D10H (IGHV3-30*02): SEQ ID NO: 4:
QVQLVESGGGVVQPGGSLRLSCAASGFTFSSYGMETHWVRQAPGKGLEWV
AFIWYDGNNKYYVDSVRGRFTISRDNFKNMETLYLQMETTGLRGEDTAVY
YCAKDLRATAAAWFGVPSVWGQGVLVTVSS;
(e) 2.46 F4H (IGHV4-39*07): SEQ ID NO: 5:
QVQLLESGPGQVKPSETLSLTCTVSGASITCDSCNWDWIRQSPGKGLEWI
GNHIFYSGRTYHKYNPSLESRVTIAVDTSKNQFSLRLTAVTAADTAVYYC
ARTVHGVATDRWGQGTLVTVSS;
(f) 2.51 B6H (IGHV3-48*01): SEQ ID NO: 6:
EVQLVESGGDLVKPGGSLKLSCAASGFTFSSYGMSWVRQTPDKRLEWVAT
ISSGGSYTYYPDSVKGRFTISRDNAKNTLYLQMSSLKSEDTAMYYCARHT
MRKCYCELKLKPPAAAPGNVHRLQKGEFQHWGQGTLVTVSS;
(g) 2.53 C10H (IGHV3-49*04): SEQ ID NO: 7:
EVQLVESGGGLIQPGRSLKLSCTASGFTFGDYVMETGWVRQAPGKGLEWV
GFIRSKAYGGTIAGTTEYAASVRGRFTISRDDSKSIAYLQMETNSLKTED
TAVYYCTRGSCSITSCAPEVLYGMETDVWGQGTTVTVSS;
(h) 2.65 F3H (IGHV3-9*01): SEQ ID NO: 8:
EVQLVESGGGSVQPGRSLRLSCAASGFTFDDYAMQWVRQAPGKGLEWVAG
LSWNGGTIGYADSVKGRFTVSRDNAKNSLYLQMNSLRAEDTALYYCVKDM
RYNWNAGLDYWGQGTLVTVSS;
(i) 2.72 D3H (IGHV3-30*02): SEQ ID NO: 9:
QVQLVESGGGVVQPGGSLRLSCAASGFPFSSFGLHWVRQAPGKGLEWVSF
ISYDGTNQYYGDFVRGRFTISRDNSKNTVFLQMNSLRAEDTAVYYCAKET
ITMVPGSFAHYVDFWGKGTTVTVSS;
(j) 2.74 E4H (IGHV4-30-4*01): SEQ ID NO: 10:
QVQLQESGPGLVKPSETLSLTCTASGGSISSSDYYWGWIRQSPGKGLEWI
ANIYYGGSTYYNPSLRSRVSISIDTSKNQFSLQMGSLTAADTAIYYCARL
NDITVVGPWDKWGPGTLVTVSS;
(k) 2.75 B3H (IGHV1-18*01): SEQ ID NO: 11:
10:QVQLQESGPGLVKPSETLSLTCTVSGSSISNYYWSWIRQSPGKGLEW
IGFIYYGGNTKYNPSLKSRVTISQDTSKSQVSLTMSSVTAAESAVYFCAR
ASCSGGYCILDYWGQGTLVTVSS;
(l) 2.77 B10H (IGHV4-59*08): SEQ ID NO: 12:
QVQLVQSGGGVVQPGRSLRLSCAASGFTFSTYGMHWVRQAPGKGLEWVAV
ISYDGNYKYYADSVKGRFTISRDNSKNTLYLEMNSLRTEDTALYYCAKDS
QLRSLLYFDWLSQGYFDHWGQGTLVTVSS;
(m) 2.81 G5H (IGHV4-39*01): SEQ ID NO: 13:
QVQLVESGPGLVKPSETLSLTCTASGGSISSSDYYWGWIRQSPGKGLEWI
ANIYYGGSTYYNPSLRSRVSISIDTSKNQFSLQMGSLTAADTAIYYCARL
NDITVVGPWDKWGPGIQVTVSS;
(n) 2.86 D7H (IGHV3-30*09): SEQ ID NO: 14:
QVQLVQSGGGVVQPGRYLRLSCAASTFTFSSNNMWVRQAPGKGLEWVALI
SYDGRISRDKSKKTVYLQMSSLRDEDTAVYKAGSTVKKRDMMKTREMINC
FDPWGRGTLVTVSS (**no CDR3);
(o) 2.92 G6H (IGHV3-9*01): SEQ ID NO: 15:
EVQLVESGGGLVQPGRSLRLSCVASGFTFGDYAMHWVRQGPGKGLEWVSG
INGNSDSVGYADSVKGRFTVSRDNAKNSLYLQLNSLTVEDTALYYCAKDL
SWGEAFDIWGQGTMVTVSS;
(p) 2.92 G8H (IGHV3-49*04): SEQ ID NO: 16:
EVQLVESGGGLVQPGRSLRLSCATSGFTFYDYAMYWVRQAPGKGLEWVGF
IRSQRYGGTSEYAASVKGRFTISRDDSKTIVYLQMNSLQAEDTAVYYCTR
GSYRCTLTACYPGYLDYWGQGTLVTVSS;
(q) 2.99 E8H (IGHV4-61*02): SEQ ID NO: 17:
QVQLVQESGPGLVKPSQTLSLTCTVSGGSVNSGAYSWNWIRQPAGKGLEW
IGRIDGRGSTKYNPSLKSRVTMSIDTSNKQFSLKLTSVTAADTAVYYCAT
TAVRSKFGVIVQNAYWFDPWGQGTLVTVSS;
(r) 2.100 E4H (IGHV3-30*07): SEQ ID NO: 18:
QVQLLESGGGVVQPGRSLRLSCTASGFTFSSYAMHWVRQAPGKGLEWVAV
MSSDGKNKYYADSVKGRFTVSRDNSKNTLYLQMDSLRPEDTAVYYCAREG
KIESGELDYYFGMDVWGQGTTVTVSS;
(s) 2.100 G3H (IGHV4-61*02): SEQ ID NO: 19:
QVQLVESGPGLVKPSQTLSLTCTVSGDSISGGRYYWSWLRQPAGKGLEWI
GRIHASGRTKYKPSLESRVTISVDTSNNQFSLKLTSLTAADTAVYYCARG
PTPYTYDSGGLYYEEYFQSWGQGTLVTVSS;

a functional fragment of any of (a) to (s). Also encompassed by the present invention are nucleic acid sequences encoding these immunoglobulin variable polypeptides and/or CDR peptides. Such sequences may be DNA or RNA (e.g., mRNA), and may be all or in part synthetic and/or recombinantly produced.

Such engineered immunoglobulins may alternatively or additionally contain, e.g., an anti-AAV kappa light chain CDR1, CDR2, and/or CDR3, identified sequentially by bold and underlining, or the kappa chain variable sequence selected from:

(a) 2.15 G3K (IGKV41*01): SEQ ID NO: 20:
DIQMTQSPFSLAVSLGDRATINCKSSQTVFFSYNNKNSVAWYQQKPGQPP
KLLIYWASTRVSGVPERFSGSDSGTDFTLTISSLHAEDVAVYYCQQYFTN
SPTFGQGTKVEIK;
(b) 2.26 F4K (IGKV228*01): SEQ ID NO: 21:
DIVMTQSPLSLAVTPGEPASISCRSSQSLLQSNGYNYLDWYLQKPGQSPQ
LLIYWGSNRASGVPDRFSGSGSGTDFTLKITRVEAEDVGVYYCMQALQTP
LTFGQGTKVEIK;
(c) 2.46 D10K (IGKV15*03): SEQ ID NO: 22:
DIQMTQSPFTLSASVGDRVTITCRASQPIDKWLAWFQQKPGKAPNLLIYK
ASTLDSGVPSRFSGSGSGTEFTLTISSLQPDDFATYYCQQYNRYWTFGPG
TKVEIK;
(d) 2.46 F4K (IGKV229*02): SEQ ID NO: 23:
DIVMTQSPLSLSVTPGQPASIFCKSNQSLLHNDDKTYLYWYLQKPGQSPH
LLIYELSSRFSGVPDRFSGSGSGTDFTLRISRVEAEDVGIYYCMQGIMLP
PTFGQGTKVEIK;
(e) 2.47 D11K (IGKV311*01): SEQ ID NO: 24:
EIVLTQSPFTLSLSPGETATLSCRASKSVSIYLAWYQQKPGQAPRLLIYD
ASNRATGIPARFSGSGSGTVFTLTITSLEPEDSAVYFCQHRDNWRGTFGP
GTKVEIK;
(f) 2.55 B4K (IGKV15*01): SEQ ID NO: 25:
DIQMTQSPFSLSASVGDRVTITCRASQPIDKWLAWFQQKPGKAPNLLIYK
ASTPDSGVPSRFSGSGSGTEFTLTIGSLQPDDFATYYCQQYNRYWTFGPG
TKVEIK;
(g) 2.92 G6Kc3 (IGKV113*02): SEQ ID NO: 26:
AIQLTQSPSSLSASVGDRVTITCRASQGISSALTWYQQKPGKTPKLLIYD
ASRLESGVPSRFSGSASGTDFTLTISSLQPEDFATYYCQHFNTFPLTFGG
GTKVEIK;
(h) 2.92 G6Kc9 (IGKV311*01): SEQ ID NO: 27:
EIVLTQSPSTLSLSPGETATLSCRASKSVSIYLAWCQQKPGQAPRLLIYD
ASNRATGIPARFSGSGSGTVFTLTITSLEPEDSAVYFCQHRDNWRGTFGP
GTKVEIK;

or a functional fragment of any of (a) to (h). Also encompassed by the present invention are nucleic acid sequences encoding these immunoglobulin variable polypeptides and/or CDR peptides. Such sequences may be DNA or RNA (e.g., mRNA), and may be all or in part synthetic and/or recombinantly produced.

It is notable that the sequence (h) identified above (2.92 G6Kc9) contains a C in the variable region, as this amino acid is rarely found in an immunoglobulin.

In still another embodiment, an engineered immunoglobulin may alternatively or additionally contain an anti-AAV lambda light chain comprising any of (a) to (i) below, and/or one or more CDR1, CDR2 and/or CDR3 from any of these sequences, which are identified sequentially by bold and underline.

(a) 2.51 B6L (IGLV1-40*01): SEQ ID NO: 28:
QSALTQPPSVSGAPGQRVTISCTGSSSNIGAGYDVHWYQQLPGTAPKLLI
YGNNNRPSGVPDRFSGSKSGTSASLAITGLQAEDEADYYCQSYDSTCYVF
GTGTKVTVL;
(b) 2.53 C10L (IGLV1-47*01): SEQ ID NO: 29:
QSVLTQPPSASGTPGQRVTISCSGRYSNIGSNYVYWYQQLPGTAPKLLIY
RNNERPSGVPDRFSGSRSGTSASLAISGLRSEDEADYYCAAWDDSLSGGV
FGGGTKLTVL;
(c) 2.65 F3L (IGLV1-40*01): SEQ ID NO: 30:
QSVLTQPPSVSGAPGQRVTISCTGSSSNIGAGYDVHWYQQLPGTAPKLLI
YGNNNRPSGVPDRFSGSKSGTSASLAITGLQAEDEADYYCQSYDSTCYVF
GTGTKVTVL;
(d) 2.77 B10L (IGLV2-11*01): SEQ ID NO: 31:
QSALTQPRSVSGSPGQSVTISCTGTSSDVGGYNYVSWYQQHPGKAPKLMI
YAVSKRPSGVPDRFSGSKSGNTASLTISGLQAEDEADYYCCSYVASYTFW
VFGTGTKVTVL;
(e) 2.81 G5L (IGLV1-40*01): SEQ ID NO: 32:
QSVLTQPPSVSGAPGQRISISCTGTSSNIGEGYDVHWYQKIPGRAPKLLI
YGNFNRPSGVPDRFSGSKSGTSASLTITGLQAEDEADYYCQSYDISLTVI
FGGGTKVTVL
(f) 2.86 D7L (IGLV2-11*01): SEQ ID NO: 33:
QSALTQPRSVSGSPGQSVTISCTGTSSNVGDYKYVSWYQQHPGKAPKLII
YNVSKRPAGVPNRFSGSKSGNTASLTISGLQADDEADYYCSSYAGSNSLN
VIFGGGTKLTVL;
(g) 2.92 G6L (IGLV2-11*01): SEQ ID NO: 34:
QSALTQPRSVSGSPGQSVTISCTGNSSDIGGYNFVSWYQQHPGKVPKLII
FDVNKRPSGVPDRFSGSRSANTASLTISGLQAEDEADYYCCSYAGSDTSE
RAVFGGGTKVTVL;
(h) 2.100 E4L (IGLV3-9*01): SEQ ID NO: 35:
SYELTQPLSVSVALGQTARITCGGNNIGSKNVHWYQQKPGQAPVLVIYRD
NNRPSGIPERFSGSNSGNTATLTISRAQAGDEAEYYCQVWDSRIYVFGSG
TKVTVL;
(i) 2.100 G3L (IGLV3-1*01): SEQ ID NO: 36:
SYELTQPPSVSVSPGQTANITCSGDKLVDKYVCWYQVRPGQSPVLVIYSD
KKRPSGIPERISGSNSGNTATLTISGSQAMDEADYYCQAWDSSIVVFGGG
TKLTVL;

or a functional fragment of any of (a) to (g). Also encompassed by the present invention are nucleic acid sequences encoding these immunoglobulin variable polypeptides and/or CDR peptides. Such sequences may be DNA or RNA (e.g., mRNA), and may be all or in part synthetic and/or recombinantly produced.

In certain embodiments, an immunoglobulin (e.g., antibody) may have at least the CDRs of a heavy chain and a light chain from the same source. In certain embodiments, a heavy chain variable domain and a light chain variable domain are from the same source. For example, an antibody may have at least the CDRs of a G3 heavy chain (G3H) variable [SEQ ID NO: 1] and/or a G3K (kappa) variable sequence [SEQ ID NO:20] and/or a G3L (lambda) variable sequence [SEQ ID NO: 36]. In another embodiment, an antibody has at least the CDRs of the D10H variable chain [SEQ ID NO: 4] and/or a D10K variable chain [SEQ ID NO: 22]. In another embodiment, an antibody has at least the CDRs of a F4H variable chain [SEQ ID NO:5] and/or a F4K variable domain [SEQ ID NO:23]. In another alternative, an antibody has at least the CDRs of a B6H variable domain [SEQ ID NO:6] and/or a B6L variable domain [SEQ ID NO: 28]. In another alternative, an antibody has at least the CDRs of a C10H variable domain [SEQ ID NO: 7] and/or a C10L variable domain [SEQ ID NO: 29]. In a further embodiment, an antibody has at least the CDRs of F3H [SEQ ID NO: 8] and/or F3L [SEQ ID NO: 30]. In another embodiment, an antibody has at least the CDRs of E4H [SEQ ID NO: 10 or SEQ ID NO:18] and/or E4L [SEQ ID NO: 35]. In another embodiment, an antibody has at least the CDRs of B10H [SEQ ID NO: 12] and/or B10L [SEQ ID NO: 31]. In another embodiment, an antibody has at least the CDRs of G5H [SEQ ID NO: 13] and/or G5L [SEQ ID NO: 32]. In yet another embodiment, an antibody has at least the CDRs of D7H [SEQ ID NO: 14] and/or D7L [SEQ ID NO: 33]. In another embodiment, an antibody has at least the CDRs of G6H [SEQ ID NO: 15] and/or G6L [SEQ ID NO: 34]. In another embodiment, an antibody has at least the CDRs of B10H [SEQ ID NO: 12] and/or B10L [SEQ ID NO: 31]. In one or more of these embodiments, the heavy chain variable domain and/or the light chain variable domain are from the same source. In certain embodiments, a full-length heavy chain or a full-length light chain are derived from the same source. Still other combinations of the heavy and/or light chains can be generated.

As described herein, the sequences isolated and/or engineered as proved herein may be used to generate artificial immunoglobulins or functional fragments thereof. Such immunoglobulins may contain a heterologous sequence, e.g., one or more constant regions from a different antibody source, a light chain from a different antibody source, a heavy chain from a different antibody source.

The invention also provides a non-naturally occurring human immunoglobulin comprising an immunoglobulin heavy chain and/or light chain consensus sequence generated according to the method provided herein. This consensus sequence may be used in an engineered antibody, or both the heavy chain and light chain of an antibody may be used to generate a non-naturally occurring human anti-AAV antibody.

The invention also provides nucleic acids encoding the immunoglobulins described herein. Once generated using the method provided herein, the immunoglobulin (e.g., heavy and/or light chain(s)) may be synthesized. Methods for sequencing a protein, peptide, or polypeptide (e.g., as an immunoglobulin) are known to those of skill in the art. Once the sequence of a protein is known, there are web-based and commercially available computer programs, as well as service based companies which back translate the amino acids sequences to nucleic acid coding sequences. See, e.g., backtranseq by EMBOSS, http://www.ebi.ac.uk/Tools/st/; Gene Infinity http://www.geneinfinity.org/sms/sms_backtranslation.html); ExPasy (http://www.expasy.org/tools/). In one embodiment, the RNA and/or cDNA coding sequences are designed for optimal expression in human cells.

The term “heterologous” when used with reference to a protein or a nucleic acid indicates that the protein or the nucleic acid comprises two or more sequences or subsequences which are not found in the same relationship to each other in nature. For instance, the nucleic acid is typically recombinantly produced, having two or more sequences from unrelated genes arranged to make a new functional nucleic acid. For example, in one embodiment, the nucleic acid has a promoter from one gene arranged to direct the expression of a coding sequence from a different gene. Thus, with reference to the coding sequence, the promoter is heterologous. The term “heterologous light chain” is a light chain containing a variable domain and/or constant domain from an antibody which has different target specificity from the specificity of the heavy chain.

The two or more ORF(s) carried by the nucleic acid molecule packaged within the vector may be expressed from two expression cassettes, one or both of which may be bicistronic. Because the expression cassettes contain heavy chains from two different antibodies, it is desirable to introduce sequence variation between the two heavy chain sequences to minimize the possibility of homologous recombination. Typically there is sufficient variation between the variable domains of the two antibodies (VH-Ab1 and VH-Ab2). However, it is desirable to ensure there is sufficient sequence variation between the constant regions of the first antibody (Ab1) and the second antibody. In one embodiment, variation in the sequence of these regions is introduced in the form of synonymous codons (i.e., variations of the nucleic acid sequence are introduced without any changes at the amino acid level). For example, the second heavy chain may have constant regions which are at least 15%, at least about 25%, up to about 30% divergent (i.e., at least about 70% to about 85%, or more, identical) over CH1, CH2 and/or CH3.

As used herein, an “expression cassette” refers to a nucleic acid molecule which comprises an immunoglobulin gene(s) (e.g., an immunoglobulin variable region, an immunoglobulin constant region, a full-length light chain, a full-length heavy chain or another fragment of an immunoglobulin construct), promoter, and may include other regulatory sequences therefor, which cassette may be delivered via a genetic element (e.g., a plasmid) to a packaging host cell and packaged into the capsid of a viral vector (e.g., an AAV or other parvovirus particle) or the envelope of an enveloped virus. Typically, such an expression cassette for generating a viral vector contains the immunoglobulin sequences described herein flanked by packaging signals of the viral genome and other expression control sequences.

In one embodiment, the nucleic acid sequences encoding the anti-AAV immunoglobulins, or functional fragments thereof, described herein are engineered into any suitable genetic element, e.g., naked DNA, phage, transposon, cosmid, episome, etc., which transfers the immunoglobulin sequences carried thereon to a host cell, e.g., for generating viral vectors in a packaging host cell and/or for delivery to a host cells in subject. The vectors provided herein may contain 1, 2, 3 or 4 open reading frame (ORF) for ten immunoglobulin domains. In one embodiment, the genetic element is a plasmid. The selected genetic element may be delivered by any suitable method, including transfection, electroporation, liposome delivery, membrane fusion techniques, high velocity DNA-coated pellets, viral infection and protoplast fusion. The methods used to make such constructs are known to those with skill in nucleic acid manipulation and include genetic engineering, recombinant engineering, and synthetic techniques. See, e.g., Green and Sambrook, Molecular Cloning: A Laboratory Manual, Cold Spring Harbor Press, Cold Spring Harbor, N.Y. (2012).

The term “amino acid substitution” and its synonyms described above are intended to encompass modification of an amino acid sequence by replacement of an amino acid with another, substituting amino acid. The substitution may be a conservative substitution. The term conservative, in referring to two amino acids, is intended to mean that the amino acids share a common property recognized by one of skill in the art. The term non-conservative, in referring to two amino acids, is intended to mean that the amino acids which have differences in at least one property recognized by one of skill in the art. For example, such properties may include amino acids having hydrophobic nonacidic side chains, amino acids having hydrophobic side chains (which may be further differentiated as acidic or nonacidic), amino acids having aliphatic hydrophobic side chains, amino acids having aromatic hydrophobic side chains, amino acids with polar neutral side chains, amino acids with electrically charged side chains, amino acids with electrically charged acidic side chains, and amino acids with electrically charged basic side chains. Thus, a conservative amino acid substitution may involve changing a first amino acid having a hydrophobic side chain with a different amino acid having a hydrophobic side chain; whereas a non-conservative amino acid substitution may involve changing a first amino acid with an acidic hydrophobic side chain with a different amino acid having a different side chain, e.g., a basic hydrophobic side chain or a hydrophilic side chain. Still other conservative or non-conservative changes may be determined by one of skill in the art.

In still other embodiments, the substitution at a given position will be to an amino acid, or one of a group of amino acids, that will be apparent to one of skill in the art in order to accomplish an objective identified herein.

The terms “identical” or percent “identity,” in the context of two or more nucleic acids or polypeptide sequences, refer to two or more sequences or subsequences that are the same or have a specified percentage of amino acid residues or nucleotides that are the same (e.g., at least about 70% identity, 75%, 80%, 85%, 90%, 91%, 92%, 93%, 94%, 95%, 96%, 97%, 98%, 99%, or higher identity over a specified region (e.g., any one of the modified ORFs provided herein when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). As another example, polynucleotide sequences can be compared using Fasta, a program in GCG Version 6.1. Fasta provides alignments and percent sequence identity of the regions of the best overlap between the query and search sequences. For instance, percent sequence identity between nucleic acid sequences can be determined using Fasta with its default parameters (a word size of 6 and the NOPAM factor for the scoring matrix) as provided in GCG Version 6.1, herein incorporated by reference. Generally, these programs are used at default settings, although one skilled in the art can alter these settings as needed. Alternatively, one of skill in the art can utilize another algorithm or computer program that provides at least the level of identity or alignment as that provided by the referenced algorithms and programs. This definition also refers to, or can be applied to, the compliment of a sequence. The definition also includes sequences that have deletions and/or additions, as well as those that have substitutions. As described below, the preferred algorithms can account for gaps and the like. Preferably, identity exists over a region that is at least about 25, 50, 75, 100, 150, 200 amino acids or nucleotides in length, and oftentimes over a region that is 225, 250, 300, 350, 400, 450, 500 amino acids or nucleotides in length or over the full-length of an amino acid or nucleic acid sequences. Such a region of identity may further exist over a functional immunoglobulin sequence, e.g., an epitope or epitope binding region or a CDR, an immunoglobulin variable region, a full-length immunoglobulin chain, or a full-length antibody.

Typically, when an alignment is prepared based upon an amino acid sequence, the alignment contains insertions and deletions which are so identified with respect to a reference sequence and the numbering of the amino acid residues is based upon a reference scale provided for the alignment. However, any given sequence may have fewer amino acid residues than the reference scale. In the present invention, when discussing the parental sequence, the term “the same position” or the “corresponding position” refers to the amino acid located at the same residue number in each of the sequences, with respect to the reference scale for the aligned sequences. However, when taken out of the alignment, each of the proteins may have these amino acids located at different residue numbers. Alignments are performed using any of a variety of publicly or commercially available Multiple Sequence Alignment Programs. Sequence alignment programs are available for amino acid sequences, e.g., the “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

As described above, the term “about” when used to modify a numerical value means a variation of ±10%, unless otherwise specified.

As used throughout this specification and the claims, the terms “comprise” and “contain” and its variants including, “comprises”, “comprising”, “contains” and “containing”, among other variants, is inclusive of other components, elements, integers, steps and the like. The term “consists of” or “consisting of” are exclusive of other components, elements, integers, steps and the like.

In one embodiment, a solid support comprising one or more of an anti-AAV immunoglobulin is provided herein. Such a support may be used for purifying an AAV vector. A variety of solid supports are known in the art and/or can be purchased commercially. Similarly, methods of linking proteins to such supports have been described in the literature. A variety of uses for such solid supports is known to those of skill in the art.

The following examples are illustrative only and are not a limitation on the invention described herein.

### EXAMPLES

To obtain a panel of human monoclonals against AAV, 31 human donors were screened for neutralizing antibodies (NAbs) against AAV2 and AAV3B and selected the donors with a titer of 1:320 against AAV2 and a titer of 1:40 against AAV3B. Bulk switched memory B cells were obtained via magnetic bead sorting and seeded on irradiated 3T3-msCD40L cells in the presence of IL-2 and IL-21 and cultured cells for 2 weeks to promote expansion and antibody secretion. Culture supernatants were screened for AAV2 and AAV3B reactivity via ELISA; of positive clones obtained, 39% were specific for AAV2, 41% were specific for AAV3B, and the remaining 20% were reactive towards both AAV2 and AAV3B. In order to obtain heavy and light chain sequences for expression and characterization of these antibodies, nested RT-PCR has been performed based on conserved regions in the immunoglobulin gene for some anti-AAV antibodies and are currently completing PCR on the remaining clones. Of the sequences obtained thus far, the majority display a substantial amount of somatic hypermutation relevant to corresponding germline sequences; the percentage of base pair substitutions resulting in a missense mutation within the complementarity-determining regions was approximately 80%, with the frequency of mutation averaging 0.26 per residue. Mutations preferentially occurred in CDRs over framework regions, again suggesting that these antibodies have undergone maturation in response to exposure to AAV antigens. Subsequent analysis of the neutralizing capacity of these antibodies as well as the mapping of their epitopes will provide critical information for evaluating the feasibility of designing AAV variants capable of evading neutralization by anti-AAV antibodies

A. Isolation of PBMCs from Human Donor Samples

Human donor blood samples were obtained from Bioreclamation and peripheral blood mononuclear cells (PBMCs) were isolated according to R. Calcedo et al, cited above. Isolated PBMCs were frozen and stored in liquid nitrogen at approximately 1×10⁷ cells/ml/vial.

### B. Selection of Human Donor

Serum for each donor (corresponding to blood sample) was obtained from Bioreclamation. All sera were screened for neutralizing antibodies against AAV2, and some were secondarily screened against AAV3B. Screening was performed according to R. Calcedo et al, cited above. The donor with the highest titer against AAV2 (donor 7) was selected for the following experiments.

Thirty-one (31) human donor serum samples were obtained from Bioreclamation and were screened for neutralizing antibodies against a panel of AAV serotypes. Titers are reported as NAb 50, the dilution factor at which transduction is reduced by 50% relative to the positive transduction control. Screening was started at 1:5; undetectable titers are reported as <1:5. Titers reported as >1:20 require further dilution.

### C. Sorting of Switched Memory B Cells

One vial of frozen PBMCs from donor 7 was thawed and recovered cells were then sorted according to the Switched Memory B Cell Isolation Kit protocol (MACS Miltenyi Biotec, order no 130-093-617). Cells were centrifuged and resuspended in PBS, pH 7.2 with 0.5% BSA and 2 mM EDTA (buffer), chilled. 100 uL of Switched Memory B Cell Biotin-Antibody cocktail (containing biotin-conjugated anti-CD2, -CD14, -CD16, -CD36, -CD43, -CD235a, -IgM, and -IgD) was then added and incubated on rocker for 10 minutes in the cold room. Cells were then washed and resuspended in buffer, followed by the addition of 200 uL of anti-biotin MicroBeads and incubation on rocker in cold room for 15 minutes. Cells were washed and resuspended in buffer then added to an LS column that was pre-rinsed with 3 mL buffer. Flow-through containing enriched switched memory B cells was collected initial suspension and 3 washes. Cells were then counted to determine density of suspension.

### D. Expansion and Irradiation of 3T3-msCD40L Cells

3T3-msCD40L cells (catalog number 12535) were obtained from the NIH AIDS Reagent Program (for their policy on use of their reagents in commercialized products see https://www.aidsreagent.org/faq.cfm#10. Cells were thawed and expanded in DMEM with 10% FBS, 1% L-glutamine, and 0.1% gentamicin. Cells were harvested and resuspended at a density of 10e6 cells/mL in culture medium. They were then irradiated with 5000 rads (50 Gy) by an X-rad irradiator. Cells were then spun down and frozen in 1-2 mL with 35e6 cells/vial and stored in liquid nitrogen.

E. Seeding of Memory B Cells with 3T3-msCD40L Cells

Five vials of 35×10⁶ 3T3-msCD40L cells were thawed and each resuspended in 7.5 mL of Iscove's Modified Dulbecco's Media with Glutamax (IMDM), and 15 μL of benzonase was added. Cells were incubated for 15 s then spun down and resuspended in 10 mL IMDM. A cell/media mixture was made for 100 96-well plates first by adding 17500 U IL-2, 87.5 μg IL-21, and 1.75×10⁸ irradiated 3T3-msCD40L cells (50 mL total) to 1680 mL complete IMDM. For each 96-well plate, the outer rows and columns were filled with 250 μL sterile H₂O to prevent evaporation. 250 μL of 3T3-msCD40L, IL-2, IL-21 in complete IMDM was added to the remaining wells in column B to act as an antibody-negative control on each plate. Sorted B cells were then added to the remaining volume to a density of 8 cells/mL to achieve a seeding density of 2 cells/well. This final mixture was then added to remaining wells of the 100 96-well plates at a volume of 250 μL/well. Seeded plates were incubated at 37° C. and 5% CO₂ for up to 14 days (cells will begin to die after 15 days). Colonies of expanding B cells may be observed as early as day 10, and supernatants may be screened for total Ab production as early as day 12 via Protein A ELISA. After 14 days, all remaining supernatant was carefully removed and frozen at −80 for future screening. 20 μL of lysis buffer (2 mL of 1M Tris-HCl pH 8.0, 1.7 mL RNAse inhibitor, NEB cat no M0314L, per 132 mL DEPC-treated H₂0) was added to each well containing B cells and plates were frozen and stored at −80° C.

### F. Screening and Selection of Clones

AAV vector particles (in this case, either AAV2 or AAV3B; each well was separately screened for both serotypes) were coated onto 96-well ELISA plates at a concentration of 1.43×10¹⁰ GC/mL (70 μL, 1×10⁹ GC/well) and incubated overnight at 4 degrees. Following overnight incubation, coating solution was discarded and plates were incubated in 3% BSA in PBS, 200 μL/well for 2 hrs at room temperature. Plates were washed 3× with 0.05% Tween in PBS, and 70 μL of B cell culture supernatant was added and incubated at 37° C. for 1 hour. Plates were washed 3× and incubated with goat anti-human antibody (1:10,000 in PBS) at room temperature for 1 hour. Plates were washed 3× and incubated with streptavidin-HRP (1:30,000 in PBS) for 1 hour. Plates were washed 3× and incubated with 150 μL/well TMB solution for 30 min at room temperature in the dark followed by quenching with sulfuric acid. Plates were read at 450 nm and 540 nm, with the absorbance at 540 nm being subtracted from that at 450 nm to determine final absorbance. Wells whose supernatants generated absorbance above the background absorbance from the 3T3-msCD40L cell-only wells were determined to be positive hits.

### G. PCR Amplification of Immunoglobulin (Ig) Loci of Positive Clones

Plates containing positive wells were thawed on ice. Once thawed, contents were mixed by pipetting up and down. For reverse transcription, 4 μL of cell lysis solution were added to 3.5 μL of RHP mix containing 2.35 μL nuclease-free water, 0.5 uL random hexamer primers (300 ng/μL, pd(N)6 Roche Applied Science), 0.5 μL Igepal CA-630 (10% solution), and 0.15 μL RNAsin on ice. Incubate for 1 min at 68 degrees and place back on ice. At 7 μL reverse transcription mix containing 3 μL First Strand buffer, 2.05 uL nuclease-free water, 1 μL DTT (100 mM stock), 0.5 μL dNTP (25 mM stock of each nucleotide) 0.2 μL RNAsin, and 0.25 μL SuperScript III. Reverse transcription was performed at 42 degrees C. for 5 min, 25 degrees C. for 10 min, 50 degrees C. for 60 min, and 94 degrees C. for 5 min.

Ig genes were amplified according to the protocol and utilizing the primers described in Wardemann 2013, cited above. The following tables provide the primers used for the 1^{st} PCR. First PCR was performed by the preparation of a master mix containing 34.16 μL H₂O, 4 μL PCR buffer, 0.13 μL of each (5′ and 3′) first primer mix, 0.4 μL dNTP solution (25 mM each nucleotide), and 0.18 μL HotStarTaq (Qiagen) for each Ig gene, heavy, kappa light and lambda light variable regions. 1 μL of cDNA for each clone was added to 39 μL of each master mix for heavy, kappa, and lambda. PCR was performed at 94° C. or 15 min, followed by 50 cycles at 94° C. for 30 s, 58° C. for 30 s (for heavy and kappa) or 60° C. for 30 s (lambda), 72° C. for 55 s, with a final step of 72° C. for 10 min. Second PCR was performed by preparing a master mix of 31.66 μL H₂O, 4 μL PCR buffer, 0.13 μL of each (5′ and 3′) second PCR primer mix, 0.4 μL dNTP (25 mM each nucleotide), and 0.18 μL HotStarTaq (Qiagen). 3.5 μL of the corresponding heavy, kappa, or lambda first PCR product was added to 36.5 μL of master mix, and PCR was performed at 94° C. for 15 min, 50 cycles of 94° C. for 30 s, 58° C. for 30 s (for heavy and kappa) or 60° C. or 30 s (lambda), 72° C. for 45 s, followed by a final step of 72° C. for 10 min. Second PCR products were analyzed by 1% agarose gel in TAE for the presence of a 450 bp (heavy), 510 bp (kappa light), or 405 bp (lambda light) band.

### H. Cloning of Ig Genes

Bands determined to be of the appropriate size by agarose gel were cut from the gel and extracted according to the protocol described in the Qiagen QIAquick™ Gel Extraction kit. The resulting DNA was then cloned into a TOPO vector and transformed into TOP10 competent cells using the TOPO-TA™ cloning kit from Life Technologies (Catalog No. K450001). Following overnight incubation at 37° C., single bacterial colonies were selected for growth and plasmid isolation. Isolated plasmid was analyzed for the presence of an Ig gene insert by EcoRI digestion followed by gel electrophoresis to confirm presence of band of desired size. Clones containing appropriately-sized inserts were sequenced using the M13 site present within the TOPO vector.

### I. Ig Sequence Analysis and Expression

Sequences obtained from TOPO clones were run through the Ig BLAST database (http://www.ncbi.nlm.nih.gov/igblast/) to determine the most closely-related germline sequence.

The following Table 1 contains the well IDs for clones that screened positive for binding to at least one serotype (AAV2 or AAV3B). Their corresponding germline loci are also listed. For those wells from which more than one variable chain sequence was identified, both germlines are listed. In these cases, all identified sequences were cloned and possible pairs expressed to determine which clone is the true hit.

TABLE 1

| Well | Heavy Chain | Light Chain | AAV3B | AAV2 | rh10 | AAV9 | AAV8 |
|---|---|---|---|---|---|---|---|
| 2.15 G3 | \|IGHV1-46*01 | \|IGKV4-1*01 | yes | yes | yes | yes | yes |
| 2.22 C8 | \|IGHV1-18*01 | | | | | | |
| 2.26 F4 | \|IGHV3-49*04 | \|IGKV2- | | | | | |
| | | 28*01 | | | | | |
| 2.46 C11 | \|IGHV3-48*01 | \|IGKV1- | yes | yes | yes | yes | Yes |
| | | 13*02 | | | | | |
| 2.46 D10 | \|IGHV3-30*02 | \|IGKV1-5*03 | yes | yes | yes | yes | Yes |
| 2.46 F4 | \|IGHV4-39*07 | \|IGKV2- | yes | yes | yes | yes | Yes |
| | | 29*02 | | | | | |
| 2.47 D11 | \|IGHV3-30*02 | \|IGKV3- | yes | yes | yes | yes | yes |
| | | 11*01 | | | | | |
| 2.51 B6 | \|IGHV3-48*01 | \|IGLV1- | yes | yes | yes | yes | Yes |
| | | 40*01 | | | | | |
| 2.53 C10 | \|IGHV1-46*03 | \|IGKV3- | yes | yes | yes | yes | yes |
| | | 11*01 | | | | | |
| | \|IGHV3-49*04 | \|IGLV1- | yes | yes | yes | yes | yes |
| | | 47*01 | | | | | |
| 2.55 B4 | | \|IGKV1-5*01 | | | | | |
| 2.56 F7 | \|IGHV3-21*04 | | | | | | |
| 2.65 F3 | \|IGHV3-9*01 | \|IGLV1- | yes | yes | yes | yes | Yes |
| | | 40*01 | | | | | |
| 2.72 D3 | \|IGHV3-30*02 | \|IGKV3- | yes | yes | yes | yes | Yes |
| | | 11*01 | | | | | |
| 2.74 E4 | \|IGHV4-30- | \|IGKV3- | | | | | |
| | 4*01 | 11*01 | | | | | |
| 2.75 B3 | \|IGHV1-18*01 | | | | | | |
| | \|IGHV4-59*08 | | | | | | |
| 2.77 B10 | \|IGHV3-33*03 | \|IGLV2- | yes | yes | yes | yes | Yes |
| | | 11*01 | | | | | |
| | \|IGHV4-59*08 | | | | | | |
| 2.81 C10 | \|IGHV3-30*02 | \|IGLV2- | | | | | |
| | | 23*03 | | | | | |
| 2.81 G5 | \|IGHV4-39*01 | \|IGLV1- | yes | yes | yes | yes | yes |
| | | 40*01 | | | | | |
| 2.86 D7 | \|IGHV3-30*09 | \|IGKV3- | | | | | |
| | | 11*01 | | | | | |
| | | \|IGLV2- | yes | yes | yes | yes | yes |
| | | 11*01 | | | | | |
| 2.91 F6 | | \|IGLV2- | | | | | |
| | | 14*01 | | | | | |
| 2.92 G6 | \|IGHV3-9*01 | \|IGKV1- | yes* | yes* | yes*, | yes*, | yes** |
| | | 13*02 | | | yes*** | yes*** | |
| | \|IGHV3-30*02 | \|IGKV3- | | | | | |
| | | 11*01 | | | | | |
| | | \|IGLV2- | | | | | |
| | | 11*01 | | | | | |
| 2.92 G8 | \|IGHV3-49*04 | \|IGLV1- | | | | | |
| | | 47*01 | | | | | |
| 2.99 E8 | \|IGHV4-61*02 | | | | | | |
| 2.100 E4 | \|IGHV3-30*07 | \|IGLV3-9*01 | yes | yes | yes | yes | yes |
| 2.100 G3 | \|IGHV4-61*02 | \|IGLV3-1*01 | yes | yes | yes | yes | yes |

*3-9*01 heavy with all combinations of light
**3-9*1 heavy chain combined with 1-47*01 lambda chain and with 3-11*01 kappa chain
***3-9*1 heavy chain combined with 1-47*01 lambda chain

The consensus amino acid sequence for each clone was determined by alignment of all TOPO clones sequenced, codon-optimized for expression in human cells, and ordered from Gene Art. Sequences were also compared to germline to determine the number of silent and missense mutations in the framework (FWR) and complementarity-determining regions (CDRs) as a measure of affinity maturation.

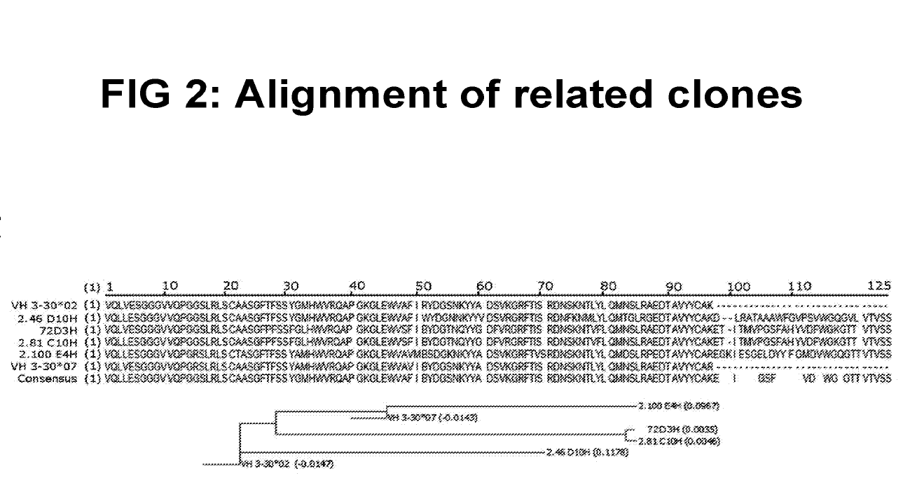

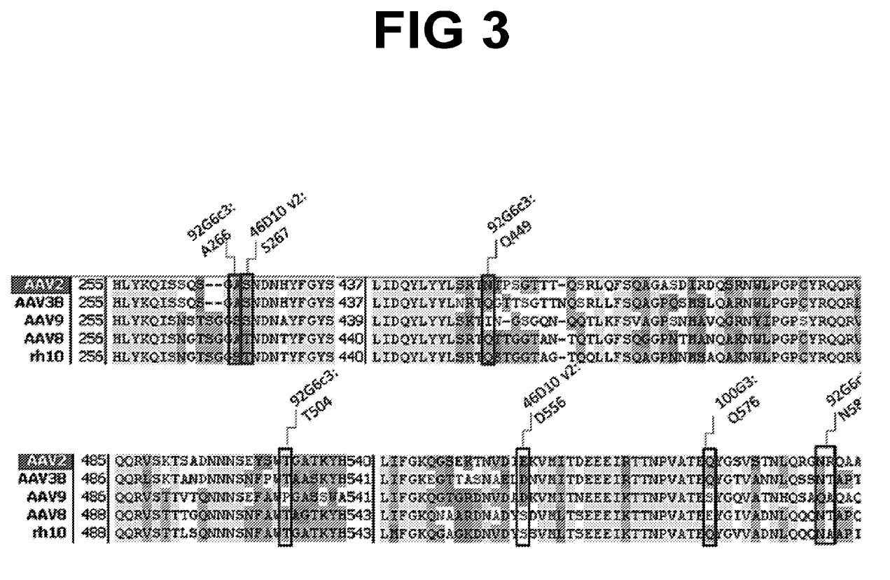

FIG. 2 provides a sample alignment of clones utilizing the same or similar germline loci. In this example, 2.46 D10H, 2.47 D11H, and 2.81 C10H all most closely align to the VH 3-30*02 germline gene. 72D3H and 2.81 C10H sequences are nearly identical, while 2.46 D10H varies at a number of positions and is more similar to the germline sequence, suggesting a lesser degree of affinity maturation. The resulting phylogenetic tree is also shown, demonstrating the relatedness of these clones.

Paired light and heavy chain variable regions were cloned into a co-expression vector with constant heavy and light chains. Paired constructs were transfected into 293 cells for expression. Binding to AAV serotypes of interest was confirmed via ELISA assay (described above) of culture supernatants. Supernatants were also evaluated for AAV neutralization by NAb assay [R. Calcedo et al, cited above]. Following this, the Fab footprint may be mapped by cryo-EM, as described in Gurda et al, cited above.

Neutralizing antibodies (NAb) against the capsid generated by prior viral infection or AAV vector administration significantly reduce not only the effective patient population but also the overall efficacy of an AAV-based gene therapy. Here, by cloning out and evaluating anti-AAV antibodies from singly-sorted memory B cells from seropositive individuals, we have designed an approach that allows us to look at the humoral immune response globally, to hone in on the immunogenic regions of the capsid itself, and to compare responses between individuals in an unbiased and therapeutically-relevant setting. In this study, we screened a panel of 30 normal human donors, selected one with high pre-existing NAb titers (1:320 for AAV2, 1:40 for AAV3B), then sorted out switched memory B cells by negative selection, seeding on irradiated ms3T3-CD40L feeder cells and culturing for 2 weeks in the presence of IL-2 and IL-21. Following supernatant screening for anti-AAV antibody production, over 100 AAV-reactive clones were identified. After isolation and cloning using nested PCR, antibodies were evaluated for AAV capsid binding as well as neutralizing capacity. To date, all antibodies demonstrated binding to AAV2 and AAV3B as well as a panel of additional AAV serotypes (8, 9, rh10, rh32.33), suggesting that AAV-binding, yet non-neutralizing antibodies may possess broad serotype specificity. To identify the epitopes for these anti-AAV antibodies, we first took a high-throughout, predictive approach. Antibody variable region sequences were placed into a generic antibody scaffold and their three-dimensional structure modeled using Kotai Antibody Builder and Rosetta Antibody followed by validation using COOT. The resulting structures were then iteratively docked onto the published structure of AAV3B using PIPER to identify the most energetically-favorable binding conformation. Thus far, the vast majority of footprint residues lie in variable regions of the capsid comprising and surrounding the 3-fold spikes. For a number of antibodies, initial prediction-directed capsid alanine scanning experiments have shown decreased antibody binding at the predicted residues, supporting the use of this approach. More comprehensive mutagenesis experiments are underway to further validate the approach and more completely map immunogenic epitopes of the AAV capsid proteins. In addition, cryo-EM analysis is currently underway for a number of these Fab-AAV complexes for additional, direct observation of the repertoire of binding footprints. These studies will provide information critical to understanding the antibody response to AAV and guide future attempts to rationally design next-generation capsids that are able to evade the anti-AAV antibody response.

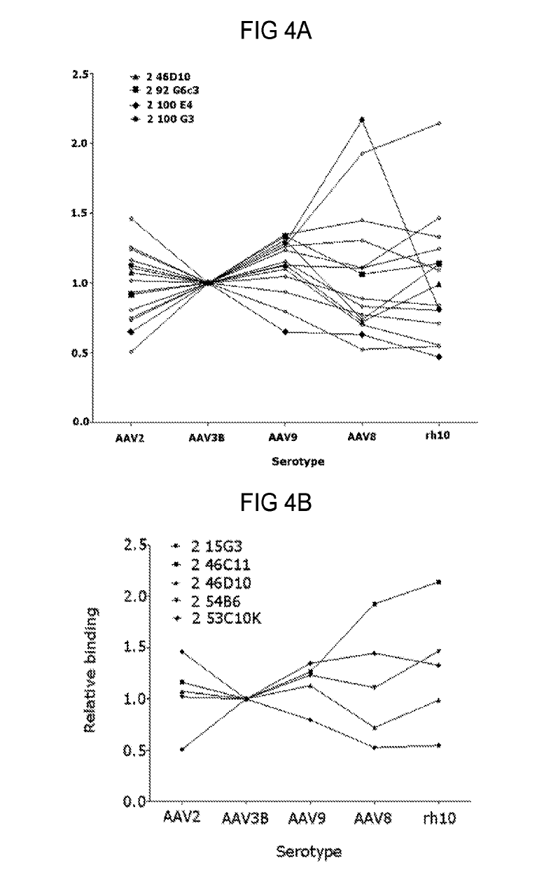

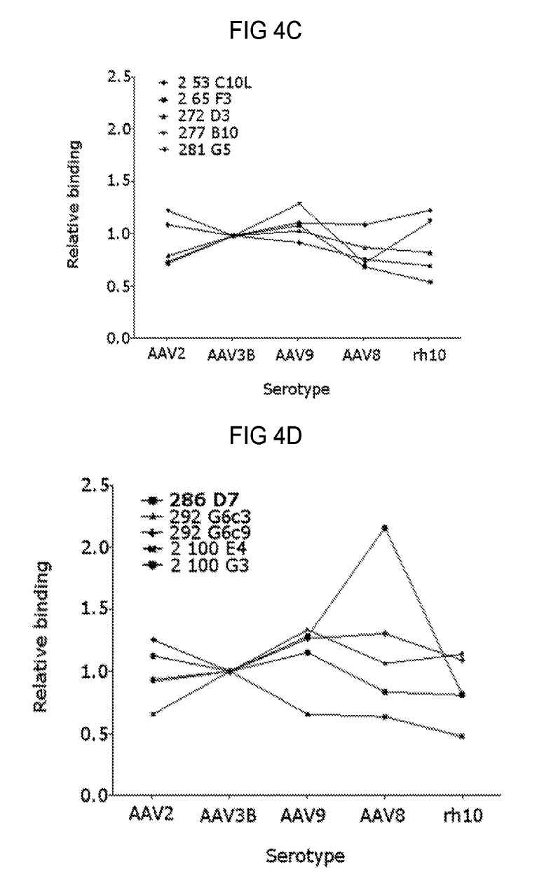

FIGS. 4A-4D show that while all recently-discovered anti-AAV antibodies demonstrate a measurable degree of binding to the serotypes tested to date, there are marked differences between antibodies in terms of their binding preferences to the individual serotypes. Some antibodies, such as 72D3, 81G5, and 86D7, show little preference for one serotype over another, while others, such as 46C11, 92G6c9, and 100G3, show a strong preference for one or two serotypes over the others. These preferences do not necessarily correlate with sequence-relatedness between serotypes.

Using the relative binding data from FIGS. 4A-4D and the epitope prediction data shown in Table 2 below a list of possible residues that could be responsible for conferring binding preference was compiled. 46D10 antibody demonstrated similar binding preference for AAV2, AAV3B, and AAV9, while demonstrating weaker binding to AAV8 and rh10. In version 1 of the epitope analysis for 46D10, position 556 was predicted to be involved in antibody-capsid interactions; AAV2, 3B, and 9 all have a negatively-charged amino acid (glutamate or aspartate), while 8 and rh10 have a polar, uncharged residue (serine). Version 2's predicted position 267 is a serine for AAV2, 3B, and 9 but a threonine for 8 and rh10, which is a less of dramatic difference in amino acid identity but directly correlates with serotype binding preferences. 92G6c3 also has a number of these residues differences predicted by binding preference. It demonstrated a preference for AAV9, with secondary preference for rh10. Both AAV9 and rh10 have a serine at position 266 while the other serotypes have an alanine (polar vs. hydrophobic side chains). AAV9 alone has an isoleucine at position 449, a hydrophobic residue, whereas all other serotypes have polar uncharged residues; the same pattern holds true at residue 504 (polar vs. hydrophobic). Finally, at position 589, AAV9 and rh10 both have the small, hydrophobic residue alanine, while the next highest binders, AAV3B and AAV8, have threonine (polar, uncharged), and the lowest binder, AAV2, has a large, charged residue (arginine), potentially disrupting antibody-capsid interactions resulting in less binding that for the other serotypes. 100G3 demonstrates a marked preference for AAV8 over the other serotypes; AAV8 has a distinctly-different residue at position 576 (glutamate, charged) whereas all other serotypes have a non-charged residue (glutamine or serine). While these observations are correlative at this time, taken together, serotype binding data and capsid sequence alignments provide additional support for the validity of the epitope predictions performed for the here-described anti-AAV antibodies. Data not shown: Many residues in each of the predictions were shared amongst all 5 serotypes evaluated (i.e., evolutionarily conserved regions of the capsid), and these regions, if they are truly involved in antibody-capsid interactions, are likely responsible for the observation that while each antibody may display a preference for one or more serotypes over others, they have demonstrated measurable binding to a number of evolutionarily-distinct AAV serotypes.

TABLE 2

| A20 | 46D10 v1 | 46D10 v2 | 46F4 | 92G6c3 | 100E4 | 100G3 | Hypervariable region |
|---|---|---|---|---|---|---|---|
| Y252 | | | | | | | I |
| N253 | | | | | | | |
| N254 | | | | | | | |
| | | K258 | | | | | |
| S261 | | | | | | | |
| S262 | | | | | | | |
| | Q263 | Q263 | | | | | |
| | S264 | S264 | | | | | |
| | G265 | G265 | | | G265 | G265 | |
| | | | | A266 | A266 | A266 | |
| | | S267 | | | | | |
| | | | N268 | N268 | N268 | N268 | |
| | D269 | | | | D269 | D269 | |
| Q374 | | | | | | | III |
| | | | | | | N382 | |
| | S384 | S384 | | | | | |
| | Q385 | Q385 | | | Q385 | | |
| L437 | | | | | | | IV |
| | R447 | R447 | | R447 | | R447 | |
| | | | Q449 | Q449 | Q449 | | |
| | | | | S453 | | | |
| | | | | Q458 | | | |
| Q469 | | | | | | | |
| | | | R488 | | R488 | | V |
| | | | | N497 | | | |
| | N498 | | | N498 | | N498 | |
| | | | | S499 | S499 | | |
| | N500 | | N500 | N500 | | N500 | |
| | | | | W503 | | | |
| | | | T504 | T504 | | | |
| | | | G513 | | | | VI |
| | | | R514 | | | | |
| | | | K528 | | | K528 | |
| | D530 | | | | D530 | D530 | |
| | | | E531 | | | | |
| | K533 | | | | | | |
| E546 | | | | | | | VII |
| T548 | | | | | | | |
| E554 | | | | | | | |
| | D556 | | | | | | |
| N557 | | | | | | | |
| | | | | | | E575 | VIII |
| | | | | | | Q576 | |
| | | | N582 | N582 | N582 | N582 | |
| | | | | N583 | N583 | | |
| | | | | | L584 | | |
| | | | S586 | | S586 | | |
| | | | N588 | N588 | N588 | | |
| | | | T589 | T589 | T589 | | |
| | | | | T592 | T592 | | |
| | | | R594 | | | | |
| | | | T595 | | | | |
| | N704 | | | | | | IX |
| | Y705 | | | | | | |
| | K707 | | | | | | |
| N710 | | N710 | | | | | |
| | | T717 | | | | | |
| N718 | | N718 | | | | | |

Table 2: Predicted epitope residues of anti-AAV antibodies on the AAV3B capsid. Residues involved in any epitope prediction are listed in the leftmost column, and antibody ID is across top row. An X indicates if a residue is predicted to be involved in the antibody-capsid interaction of the given antibody. Each group of residues is also shaded according to the corresponding hypervariable region of the AAV capsid protein VP.

Discussion: A previously-discovered antibody against AAV2, A20, was used as validation for the modeling-based epitope prediction approach. The residues listed largely agree with previously-published cryo EM data of the A20 Fab in complex with AAV2 (McCraw et al, Virology (2012) 431:40-910.1016/j.virol. 2012May 4), supporting the validity of this predictive approach used to increase the throughput of epitope mapping attempts. A number of anti-AAV antibodies described in this patent were then subjected to the same workflow, and the resulting interacting residue predictions are listed. The vast majority of predicted residues lie within the hypervariable regions of the AAV capsid, where the capsid itself has the most sequence variation as well as structural flexibility. However, capsid serotypes are largely conserved at a number of these positions, despite their inclusion in hypervariable regions. More specifically, the predicted residues lie within the 3-fold axis, on or around the 3-fold spikes on the capsid surface. Interestingly, despite all recognizing residues in a relatively confined region of the capsid, each antibody has a number of distinct residues predicted to be involved antibody-capsid interactions, suggesting that while there is overlap in the general location of antibody binding, the epitopes themselves are largely distinct. Additionally, one antibody, 46D10, was predicted to bind in two potential orientations around the 3-fold axis, listed here as version 1 and version 2.

Finally, cryo EM reconstruction of the 100G3-AAV3B complex indicates Fab density in hypervariable regions V and VIII, which was predicted by capsid-antibody docking analysis, further support for this predictive method. Additionally, preliminary site-directed mutagenesis experiments in which the predicted epitope residues for 100G3 were iteratively mutated to alanine suggest that an alanine at positions 497/98, 499/500, and/or 587/88 partially disrupts the ability of recombinant 100G3 to bind to AAV3B capsid as measured by ELISA. The epitope for 100G3 as indicated by the current cryoEM analysis is 493-ANDNNNS-499 and 586-SSNT-589, VP1 numbering of the AAV3B capsid sequence, at a resolution of approximately 13.5 angstroms.

### SEQUENCE LISTING FREE TEXT

The following information is provided for sequences containing free text under numeric identifier <223>.

| SEQ ID NO: | |
|---|---|
| (containing | |
| free text) | Free text under <223> |
| 39 | <223> Consensus VH chain |
| | <220> |
| | <221> misc_feature |
| | <222> (98)..(99) |
| | <223> Xaa can be any naturally |
| | occurring amino acid |
| | <220> |
| | <221> misc_feature |
| | <222> (101)..(104) |
| | <223> Xaa can be any naturally |
| | occurring amino acid |
| | <220> |
| | <221> misc_feature |
| | <222> (108)..(110) |
| | <223> Xaa can be any naturally |
| | occurring amino acid |
| | <220> |
| | <221> misc_feature |
| | (containing free text) |
| | <222> (113)..(113) |
| | <223> Xaa can be any naturally |
| | occurring amino acid |
| | <220> |
| | <221> misc_feature |
| | <222> (116)..(116) |
| | <223> Xaa can be any naturally |
| | occurring amino acid |

This application contains sequences and a sequence listing, which is hereby incorporated by reference. All publications, patents, and patent applications, and priority applications U.S. Provisional Patent Application No. 62/232,740, filed Apr. 17, 2016 and U.S. Provisional Patent Application No. 62/153,000, filed Apr. 27, 2015, cited in this application are hereby incorporated by reference in their entireties as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications can be made thereto without departing from the spirit or scope of the appended claims.

1. A non-naturally occurring antibody comprising an anti-AAV immunoglobulin chain comprising the sequence selected from at least one of (a), (b), and/or (c) and a sequence heterologous thereto:
(a) a heavy chain comprising a sequence selected from (i) to (xviii), or a fragment thereof comprising at least one complementarity determining region (CDR):
(i) 2.15 G3H (IGHV1-46*01): SEQ ID NO: 1:
QVQLVQSGAEVKKSGASVKVSCQASGYTFTDYWMHWVRQAPGQGLEWMGI
IDGSGGSTNSAQKFRGRLTMTRDTSTRTVYMELSSLRSDDTAVYYCARVM
TPKFTYDAFEIWGQGTVVTVSS;
(ii) 2.22 C8H (IGHV1-18*01): SEQ ID NO: 2:
QVQLVESGSEVKKPGASVKVSCKASGYDFSRHGITWVRQAPGQGLEWMET
GWISGYDGNTNYAQRLRGRVTMETTTDTSTSTVYMETELRSLRSDDTAVY
YCARDRFVEWFIVIDFWGQGTLVTVSS;
(iii) 2.46 C11H (IGHV3-48*01): SEQ ID NO: 3:
EVQLVESGGGLVQPGGSRKLSCAASGFTFSSYGMHWVRQAPEKGLEWVAY
ISSSSGTIYYADTVKGRFTISRDNAKNTLFLQMTSLRSEDTAMYYCARHT
MSNSYCELKLKPPAAAPGNVHRLQKGEFQHWGQGTLVTVSS;
(iv) 2.46 D10H (IGHV3-30*02): SEQ ID NO: 4:
QVQLVESGGGVVQPGGSLRLSCAASGFTFSSYGMETHWVRQAPGKGLEWV
AFIWYDGNNKYYVDSVRGRFTISRDNFKNMETLYLQMETTGLRGEDTAVY
YCAKDLRATAAAWFGVPSVWGQGVLVTVSS;
(v) 2.46 F4H (IGHV4-39*07): SEQ ID NO: 5:
QVQLLESGPGQVKPSETLSLTCTVSGASITCDSCNWDWIRQSPGKGLEWI
GNHIFYSGRTYHKYNPSLESRVTIAVDTSKNQFSLRLTAVTAADTAVYYC
ARTVHGVATDRWGQGTLVTVSS;
(vi) 2.51 B6H (IGHV3-48*01): SEQ ID NO: 6:
EVQLVESGGDLVKPGGSLKLSCAASGFTFSSYGMSWVRQTPDKRLEWVAT
ISSGGSYTYYPDSVKGRFTISRDNAKNTLYLQMSSLKSEDTAMYYCARHT
MRKCYCELKLKPPAAAPGNVHRLQKGEFQHWGQGTLVTVSS;
(vii) 2.53 C10H (IGHV3-49*04): SEQ ID NO: 7:
EVQLVESGGGLIQPGRSLKLSCTASGFTFGDYVMETGWVRQAPGKGLEWV
GFIRSKAYGGTIAGTTEYAASVRGRFTISRDDSKSIAYLQMETNSLKTED
TAVYYCTRGSCSITSCAPEVLYGMETDVWGQGTTVTVSS;
(h) 2.65 F3H (IGHV3-9*01): SEQ ID NO: 8:
EVQLVESGGGSVQPGRSLRLSCAASGFTFDDYAMQWVRQAPGKGLEWVAG
LSWNGGTIGYADSVKGRFTVSRDNAKNSLYLQMNSLRAEDTALYYCVKDM
RYNWNAGLDYWGQGTLVTVSS;
(viii) 2.72 D3H (IGHV3-30*02): SEQ ID NO: 9:
QVQLVESGGGVVQPGGSLRLSCAASGFPFSSFGLHWVRQAPGKGLEWVSF
ISYDGTNQYYGDFVRGRFTISRDNSKNTVFLQMNSLRAEDTAVYYCAKET
ITMVPGSFAHYVDFWGKGTTVTVSS
(ix) 2.74 E4H (IGHV4-30-4*01): SEQ ID NO: 10:
QVQLQESGPGLVKPSETLSLTCTASGGSISSSDYYWGWIRQSPGKGLEWI
ANIYYGGSTYYNPSLRSRVSISIDTSKNQFSLQMGSLTAADTAIYYCARL
NDITVVGPWDKWGPGTLVTVSS;
(x) 2.75 B3H (IGHV1-18*01): SEQ ID NO: 11:
QVQLQESGPGLVKPSETLSLTCTVSGSSISNYYWSWIRQSPGKGLEWIGF
IYYGGNTKYNPSLKSRVTISQDTSKSQVSLTMSSVTAAESAVYFCARASC
SGGYCILDYWGQGTLVTVSS;
(xi) 2.77 B10H (IGHV4-59*08): SEQ ID NO: 12:
QVQLVQSGGGVVQPGRSLRLSCAASGFTFSTYGMHWVRQAPGKGLEWVAV
ISYDGNYKYYADSVKGRFTISRDNSKNTLYLEMNSLRTEDTALYYCAKDS
QLRSLLYFDWLSQGYFDHWGQGTLVTVSS;
(xii) 2.81 G5H (IGHV4-39*01): SEQ ID NO: 13:
QVQLVESGPGLVKPSETLSLTCTASGGSISSSDYYWGWIRQSPGKGLEWI
ANIYYGGSTYYNPSLRSRVSISIDTSKNQFSLQMGSLTAADTAIYYCARL
NDITVVGPWDKWGPGIQVTVSS;
(xiii) 2.86 D7H (IGHV3-30*09): SEQ ID NO: 14:
QVQLVQSGGGVVQPGRYLRLSCAASTFTFSSNNMWVRQAPGKGLEWVALI
SYDGRISRDKSKKTVYLQMSSLRDEDTAVYKAGSTVKKRDMMKTREMINC
FDPWGRGTLVTVSS (**no CDR3);
(xiv) 2.92 G6H (IGHV3-9*01): SEQ ID NO: 15:
EVQLVESGGGLVQPGRSLRLSCVASGFTFGDYAMHWVRQGPGKGLEWVSG
INGNSDSVGYADSVKGRFTVSRDNAKNSLYLQLNSLTVEDTALYYCAKDL
SWGEAFDIWGQGTMVTVSS;
(xv) 2.92 G8H (IGHV3-49*04): SEQ ID NO: 16:
EVQLVESGGGLVQPGRSLRLSCATSGFTFYDYAMYWVRQAPGKGLEWVGF
IRSQRYGGTSEYAASVKGRFTISRDDSKTIVYLQMNSLQAEDTAVYYCTR
GSYRCTLTACYPGYLDYWGQGTLVTVSS;
(xvi) 2.99 E8H (IGHV4-61*02): SEQ ID NO: 17:
QVQLVQESGPGLVKPSQTLSLTCTVSGGSVNSGAYSWNWIRQPAGKGLEW
IGRIDGRGSTKYNPSLKSRVTMSIDTSNKQFSLKLTSVTAADTAVYYCAT
TAVRSKFGVIVQNAYWFDPWGQGTLVTVSS;
(xvii) 2.100 E4H (IGHV3-30*07): SEQ ID NO: 18:
QVQLLESGGGVVQPGRSLRLSCTASGFTFSSYAMHWVRQAPGKGLEWVAV
MSSDGKNKYYADSVKGRFTVSRDNSKNTLYLQMDSLRPEDTAVYYCAREG
KIESGELDYYFGMDVWGQGTTVTVSS;
or
(xviii) 2.100 G3H (IGHV4-61*02): SEQ ID NO: 19:
QVQLVESGPGLVKPSQTLSLTCTVSGDSISGGRYYWSWLRQPAGKGLEWI
GRIHASGRTKYKPSLESRVTISVDTSNNQFSLKLTSLTAADTAVYYCARG
PTPYTYDSGGLYYEEYFQSWGQGTLVTVSS;
(b) a kappa light chain comprising a sequence of (i) to (viii), or a fragment thereof comprising at least CDR:
(i) 2.15 G3K (IGKV41*01): SEQ ID NO: 20:
DIQMTQSPFSLAVSLGDRATINCKSSQTVFFSYNNKNSVAWYQQKPGQPP
KLLIYWASTRVSGVPERFSGSDSGTDFTLTISSLHAEDVAVYYCQQYFTN
SPTFGQGTKVEIK;
(ii) 2.26 F4K (IGKV228*01): SEQ ID NO: 21:
DIVMTQSPLSLAVTPGEPASISCRSSQSLLQSNGYNYLDWYLQKPGQSPQ
LLIYWGSNRASGVPDRFSGSGSGTDFTLKITRVEAEDVGVYYCMQALQTP
LTFGQGTKVEIK;
(iii) (2.46 D10K (IGKV15*03): SEQ ID NO: 22:
DIQMTQSPFTLSASVGDRVTITCRASQPIDKWLAWFQQKPGKAPNLLIYK
ASTLDSGVPSRFSGSGSGTEFTLTISSLQPDDFATYYCQQYNRYWTFGPG
TKVEIK;
(iv) 2.46 F4K (IGKV229*02): SEQ ID NO: 23:
DIVMTQSPLSLSVTPGQPASIFCKSNQSLLHNDDKTYLYWYLQKPGQSPH
LLIYELSSRFSGVPDRFSGSGSGTDFTLRISRVEAEDVGIYYCMQGIMLP
PTFGQGTKVEIK
(v) 2.47 D11K (IGKV311*01): SEQ ID NO: 24:
EIVLTQSPFTLSLSPGETATLSCRASKSVSIYLAWYQQKPGQAPRLLIYD
ASNRATGIPARFSGSGSGTVFTLTITSLEPEDSAVYFCQHRDNWRGTFGP
GTKVEIK;
(vi) 2.55 B4K (IGKV15*01): SEQ ID NO: 25:
DIQMTQSPFSLSASVGDRVTITCRASQPIDKWLAWFQQKPGKAPNLLIYK
ASTPDSGVPSRFSGSGSGTEFTLTIGSLQPDDFATYYCQQYNRYWTFGPG
TKVEIK;
(vii) 2.92 G6Kc3 (IGKV113*02): SEQ ID NO: 26:
AIQLTQSPSSLSASVGDRVTITCRASQGISSALTWYQQKPGKTPKLLIYD
ASRLESGVPSRFSGSASGTDFTLTISSLQPEDFATYYCQHFNTFPLTFGG
GTKVEIK;
and/or
(viii) 2.92 G6Kc9 (IGKV311*01): SEQ ID NO: 27:
EIVLTQSPSTLSLSPGETATLSCRASKSVSIYLAWCQQKPGQAPRLLIYD
ASNRATGIPARFSGSGSGTVFTLTITSLEPEDSAVYFCQHRDNWRGTFGP
GTKVEIK.
and/or (c) an anti-AAV lambda light chain comprising a sequence of one of (i) to (ix), or a fragment thereof comprising at least one CDR:
(i) 2.51 B6L (IGLV1-40*01): SEQ ID NO: 28:
QSALTQPPSVSGAPGQRVTISCTGSSSNIGAGYDVHWYQQLPGTAPKLLI
YGNNNRPSGVPDRFSGSKSGTSASLAITGLQAEDEADYYCQSYDSTCYVF
GTGTKVTVL;
(ii) 2.53 C10L (IGLV1-47*01): SEQ ID NO: 29:
QSVLTQPPSASGTPGQRVTISCSGRYSNIGSNYVYWYQQLPGTAPKLLIY
RNNERPSGVPDRFSGSRSGTSASLAISGLRSEDEADYYCAAWDDSLSGGV
FGGGTKLTVL;
(iii) 2.65 F3L (IGLV1-40*01): SEQ ID NO: 30:
QSVLTQPPSVSGAPGQRVTISCTGSSSNIGAGYDVHWYQQLPGTAPKLLI
YGNNNRPSGVPDRFSGSKSGTSASLAITGLQAEDEADYYCQSYDSTCYVF
GTGTKVTVL;
(iv) 2.77 B10L (IGLV2-11*01): SEQ ID NO: 31:
QSALTQPRSVSGSPGQSVTISCTGTSSDVGGYNYVSWYQQHPGKAPKLMI
YAVSKRPSGVPDRFSGSKSGNTASLTISGLQAEDEADYYCCSYVASYTFW
VFGTGTKVTVL;
(v) 2.81 G5L (IGLV1-40*01): SEQ ID NO: 32:
QSVLTQPPSVSGAPGQRISISCTGTSSNIGEGYDVHWYQKIPGRAPKLLI
YGNFNRPSGVPDRFSGSKSGTSASLTITGLQAEDEADYYCQSYDISLTVI
FGGGTKVTVL
(vi) 2.86 D7L (IGLV2-11*01): SEQ ID NO: 33:
QSALTQPRSVSGSPGQSVTISCTGTSSNVGDYKYVSWYQQHPGKAPKLII
YNVSKRPAGVPNRFSGSKSGNTASLTISGLQADDEADYYCSSYAGSNSLN
VIFGGGTKLTVL;
(vii) 2.92 G6L (IGLV2-11*01): SEQ ID NO: 34:
QSALTQPRSVSGSPGQSVTISCTGNSSDIGGYNFVSWYQQHPGKVPKLII
FDVNKRPSGVPDRFSGSRSANTASLTISGLQAEDEADYYCCSYAGSDTSE
RAVFGGGTKVTVL;
(viii) 2.100 E4L (IGLV3-9*01): SEQ ID NO: 35:
SYELTQPLSVSVALGQTARITCGGNNIGSKNVHWYQQKPGQAPVLVIYRD
NNRPSGIPERFSGSNSGNTATLTISRAQAGDEAEYYCQVWDSRIYVFGSG
TKVTVL;
or
(ix) 2.100 G3L (IGLV3-1*01): SEQ ID NO: 36:
SYELTQPPSVSVSPGQTANITCSGDKLVDKYVCWYQVRPGQSPVLVIYSD
KKRPSGIPERISGSNSGNTATLTISGSQAMDEADYYCQAWDSSIVVFGGG
TKLTVL.

2. The antibody according to claim 1, wherein the heterologous sequence is a heavy chain and/or light chain constant region.

3. The antibody according to claim 1, wherein the heterologous sequence is a framework region into which the fragment comprising the one or more CDRs is inserted.

4. A synthetic nucleic acid sequence encoding an immunoglobulin comprising a kappa light chain variable region according to claim 1, a lambda light chain according to claim 1, or a fragment thereof.

5. A synthetic nucleic acid sequence encoding an immunoglobulin comprising a heavy chain variable region according to claim 1 (a) and a light chain variable region of (b) or (c) according to claim 1.

6. A panel of human anti-AAV antibodies, wherein said panel comprises two or more anti-AAV antibodies according to any one of claims 1 to 5.

7. The panel according to claim 6, which comprises 3 to 25 anti-AAV antibodies directed against a single AAV.

8. A solid support comprising one or more of an anti-AAV heavy chain, kappa light chain, or a lambda light chain of claim 1.

9. A method of purifying an AAV vector comprising contacting a suspension comprising a selected AAV with the solid support of claim 8.

10. A method of generating human anti-AAV antibodies which comprises:
(a) sorting memory B cells from a sample comprising human plasma;
(b) culturing the memory B cells and screening supernatant from the cell culture for anti-AAV activity and/or binding to AAV;
(c) amplifying immunoglobulins from the cell culture supernatant selected from one or more of a heavy chain and/or a light chain, or a fragment thereof using RT-PCR;
(d) cloning sequences encoding the immunoglobulins produced from the amplified immunoglobulins of (c); and
(e) sequencing the sequences obtained from the clones and generating an immunoglobulin consensus sequence.

11. The method according to claim 10, wherein the memory B cells are sorted via magnetic beads.

12. The method according to claim 10, further comprising obtaining the amino acids encoded by the amplified immunoglobulins of (c), synthesizing an nucleic acid sequence having a sequence divergent to the amplified immunoglobulins which encode the same amino acids, wherein the synthetic immunoglobulin nucleic acid sequences are used in the cloning of (d).

13. The method according to claim 10, further comprising expressing the immunoglobulin heavy chain.

14. The method according to claim 10, further comprising co-expressing the immunoglobulin heavy chain and a light chain.

15. A panel of human anti-AAV antibodies, wherein said anti-AAV antibodies are generated according to claim 10.

16. The panel according to claim 15, which comprises 3 to 25 anti-AAV antibodies directed against a single AAV.

17. A non-naturally occurring immunoglobulin comprising an immunoglobulin consensus sequence generated according to claim 10.
